SEQUENCE LISTING (4 sequences)

SEQ ID NO: 1 - DNA, 6752 bp, Artificial Sequence
  plasmid sequence with recombinant human gene sequences within
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcacgtgggcccggtaccaccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggatctggggccaccaacttttcattgctcaagcaggcgggcgatgtggaggaaaaccctggcccctgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggatctggggccaccaacttttcattgctcaagcaggcgggcgatgtggaggaaaaccctggccccgagacagacacactcctgctatgggtactgctgctctgggttccaggttccactggtgacactcaggattgcagcttccagcattcacccatatcatcagattttgcagtaaagatcagggaactctccgattatctccttcaagactaccccgtaacagtggcctccaatttgcaagacgaagagctttgtggtgccctctggcggctcgttttggcccaaaggtggatggaacggcttaagacagtcgctggcagcaagatgcaagggttgctcgaacgagtcaatacagagatccattttgtaaccaagtgtgcatttcaaccgccgccaagctgccttcgctttgttcagacgaatataagtagactgttgcaggaaacctccgagcaactcgtagccctgaagccctggattacacggcaaaatttcagtcggtgccttgagcttcagtgtcagcctgatagtagtaccttgcctccgccatggtcccccaggcctcttgaagctacagctccgacagcccctcagccgggcagtagtggtagttctggagccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaaggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 2 - protein, 274 aa, Artificial Sequence
  human recombinant protein
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPG

SEQ ID NO: 3 - protein, 349 aa, Artificial Sequence
  recombinant human protein
PCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGATNFSLLKQAGDVEENPG

SEQ ID NO: 4 - protein, 287 aa, Artificial Sequence
  recombinant human protein
PETDTLLLWVLLLWVPGSTGDTQDCSFQHSPISSDFAVKIRELSDYLLQDYPVTVASNLQDEELCGALWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPGSSGSSGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

(19) United States
(12) Patent Application Publication
(10) Pub. No.: US 2020/0123566 A1
(43) Pub. Date: Apr. 23, 2020

(54) MULTIGENE CONSTRUCT FOR IMMUNE-MODULATORY PROTEIN EXPRESSION AND METHODS OF USE

(71) Applicant: OncoSec Medical Incorporated, Pennington, NJ (US)

(72) Inventor: David A. Canton, Poway, CA (US)

(21) Appl. No.: 16/621,823

(22) PCT Filed: Jun. 13, 2018
(86) PCT No.: PCT/IB2018/054344

§ 371 Date: Dec. 12, 2019

Related U.S. Application Data
(60) Provisional application No. 62/519,120, filed on Jun. 13, 2017.
(60) Provisional application No. 62/582,917, filed on Nov. 7, 2017.
(60) Provisional application No. 62/628,917, filed on Feb. 9, 2018.

(51) Int. Cl. C12N 15/85; C07K 14/54; A61K 38/20; C07K 14/71; A61K 38/17; A61K 48/00; A61P 35/00; A61K 41/00; C07K 14/47; A61K 39/00; A61K 45/06; A61N 1/32

(52) U.S. Cl. CPC C12N 15/85; C07K 14/5434; A61K 38/208; C07K 14/71; A61K 38/179; A61K 48/0016; C07K 2319/33; A61K 41/0047; C07K 14/4748; A61K 39/001188; A61K 45/06; A61N 1/327; C12N 2800/107; A61P 35/00

(57) ABSTRACT

Provided are expression vector constructs encoding multiple immunomodulatory proteins where each protein or component thereof can be expressed utilizing appropriate promoters and/or translation modifiers. Additional immunomodulatory proteins and genetic adjuvants containing shared tumor antigens can be added to further therapeutic potential as well as allow tracking of therapeutic treatment. Also provided are methods of use for the expression vectors.

### CROSS-REFERENCE TO RELATED APPLICATIONS

This application claims the benefit of U.S. Application No. 62/519,120, filed Jun. 13, 2017, U.S. Application No. 62/582,917, filed Nov. 7, 2017, and U.S. Application No. 62/628,917, filed Feb. 9, 2018, each of which is herein incorporated by reference in its entirety for all purposes.

### REFERENCE TO A SEQUENCE LISTING SUBMITTED AS A TEXT FILE VIA EFS WEB

The Sequence Listing filed electronically herewith is also hereby incorporated by reference in its entirety (File Name: OM1702PCT SQ_ST25.txt; Date Created: Jun. 12, 2018; File Size: 45 KB).

### FIELD

Recombinant expression vector for intratumoral delivery of three genes encoding therapeutically active multimeric and fusion polypeptides are described. Nucleic acids encoding polypeptides separated by translation modulating element are provided. Also provided are methods of delivery.

### BACKGROUND

E. coli plasmids have long been an important source of recombinant DNA molecules used by researchers and by industry. Today, plasmid DNA is becoming increasingly important as the next generation of biotechnology products (e.g., gene medicines and DNA vaccines) make their way into clinical trials, and eventually into the pharmaceutical marketplace. Expression plasmid DNA may find application as vehicles to deliver therapeutic proteins to sites in a patient where treatment is needed, e.g., tumors.

This “intratumoral delivery” often involves the delivery of immunomodulators to the tumor microenvironment. Immunotherapy has recently drawn attention as a fourth method following surgery, chemotherapy and radiation therapy for treating tumors. Since immunotherapy utilizes the immunity inherent to humans, it is said that the physical burdens on patients are less in immunotherapy than those in other therapies. The therapeutic approaches known as immunotherapies include: cell transfer therapy in which cells such as lymphokine-activated cells, natural killer T-cells or γδT cells are obtained, for example, from exogenously-induced cytotoxic T-lymphocytes (CTLs) or peripheral blood lymphocytes by expansion culture using various method are transferred; dendritic cell-transfer therapy or peptide vaccine therapy by which in vivo induction of antigen-specific CTLs is expected; Th1 cell therapy; and immune gene therapy in which genes expected to have various effects are introduced ex vivo into the above-mentioned cells to transfer them in vivo. In these immunotherapies, CD4-positive T cells and CD8-positive T cells have traditionally been known to play a critical role.

In vivo electroporation is a gene delivery technique that has been used successfully for efficient delivery of plasmid DNA to many different tissues. Studies have reported the administration of in vivo electroporation for delivery of plasmid DNA to B16 melanomas and other tumor tissues. Systemic and local expression of a gene or cDNA encoded by a plasmid can be obtained with administration of in vivo electroporation. Use of in vivo electroporation enhances plasmid DNA uptake in tumor tissue, resulting in expression within the tumor, and delivers plasmids to muscle tissue, resulting in systemic cytokine expression.

It has been shown that electroporation can be used to transfect cells in vivo with plasmid DNA. Recent studies have shown that electroporation is capable of enhancing delivery of plasmid DNA as an antitumor agent. Electroporation has been administered for treatment of hepatocellular carcinomas, adenocarcinoma, breast tumors, squamous cell carcinoma and B16.F10 melanoma in rodent models. The B16.F10 murine melanoma model has been used extensively for testing potential immunotherapy protocols for the delivery of an immunomodulatory molecule including cytokines either as recombinant protein or by gene therapy.

Various protocols known in the art can be utilized for the delivery of plasmid encoding an immunomodulatory protein utilizing in vivo electroporation for the treatment of cancer. The protocols known in the art describe in vivo electroporation mediated cytokine based gene therapy, both intratumoral and intramuscular, utilizing low-voltage and long-pulse currents.

Combination immunotherapies that involve various phases of the cancer-immunity cycle may enhance the ability to prevent immune escape by targeting multiple mechanisms by which tumor cells avoid elimination by the immune system, with synergistic effects that may offer improved efficacy in broader patient populations. Often these combination therapeutic immunomodulatory proteins are complex molecules involving one or more homo- or heterodimeric chains, e.g., IL-12, fusion proteins encoding genetic adjuvants, and tumor or viral antigens. Administration of multiple proteins as therapeutics is complex and costly. Use of intratumoral delivery of multiple encoded proteins using expression plasmids is simpler and more cost effective.

Furthermore, use of proper translation elements and optimized electroporation parameters can result in improved expression of the multiple proteins, including heterodimeric immunostimulatory cytokines, and reduce the frequency of therapeutic administration of the plasmid therapeutic. However, current expression plasmid constructs do not address the need for adequate production of each immunomodulatory protein. Described are compounds and methods of using the compounds that address this need by providing an expression vectors encoding the heterodimeric cytokine IL-12 alone and with FLT3 ligand fused to a tumor antigen with appropriately placed promoters and translation modifiers.

### SUMMARY

Described are expression vector comprising a plurality of expression cassettes defined by the formula: P-A-T-A′-T-B, wherein: a) P is a human CMV promoter; b) A and A′ are interleukin-12 (IL-12) p35 and p40, respectively; c) B is FLT3L fused to at least one antigen; and d) T is a translation modulating element such as a P2A translation modification element. In certain embodiments, the antigen is selected from the group consisting of: NYESO-1, OVA, RNEU, MAGE-A1, MAGE-A2, Mage-A10, SSX-2, Melan-A, MART-1, Tyr, Gp100, LAGE-1, Survivin, PRS pan-DR, CEA peptide CAP-1, OVA, HCV-NS3, TERT, WT1, PSMA, and an HPV vaccine peptide. In some embodiments, the expression vector is a plasmid. In some embodiments, the expression vector comprises a nucleic acid sequence of SEQ ID NO: 1, SEQ ID NO: 8, SEQ ID NO: 10, or SEQ ID NO: 12, or a nucleotide sequence having at least 70% identity to the nucleotide sequence of SEQ ID NO: 1, SEQ ID NO: 8, SEQ ID NO: 10, or SEQ ID NO: 12. In some embodiments, the expression vector encoded a polypeptide comprising the amino acid sequence of SEQ ID NO: 9 or SEQ ID NO: 10 or a polypeptide having at least 70% identity to the amino acid sequence of SEQ ID NO: 9 or SEQ ID NO: 10.

Also described are methods of treating a tumor in a subject, comprising delivery of one or more of the described expression vectors into the tumor using at least one intratumoral electroporation pulse. In some embodiments, the intratumoral electroporation pulse has a field strength of about 200 V/cm to 1500 V/cm. In other embodiments, the subject is a human. In some embodiments, the tumor can be, but is not limited to, melanoma, triple negative breast cancer, Merkel Cell Carcinoma, CTCL, and head and neck squamous cell carcinoma (HNSCC). In another embodiment, the electroporation pulse is delivered by a generator capable of electrochemical impedance spectroscopy.

Methods are described for treating a tumor in a subject comprising at least one low voltage intratumoral electroporation (IT-EP) treatment delivering an any of the described expression vectors encoding interleukin-12 (IL-12) containing at least one P2A exon skipping motif. In some embodiments, the IT-EP is at a field strength of 200 V/Cm to 500 V/cm and a pulse length of about 100 μs (microsecond) to about 50 ms (millisecond). In further embodiments, the treatment comprises at least one IT-EP treatment at a field strength of at least 400 V/cm and a pulse length of about 10 ms. Also contemplated is wherein the low voltage IT-EP treatment of the IL-12 encoded plasmid containing P2A comprises at least one of the following when compared to an IL-12 encoded plasmid containing an IRES motif: a) at least 3.6 times higher intratumoral expression of IL-12; b) a lower mean tumor volume in a treated tumor lesion; c) a lower mean tumor volume in an untreated contralateral tumor lesion; d) a higher influx of lymphocytes into the tumor; e) an increase of circulating tumor-specific CD8+ T cells; f) an increase of lymphocyte and monocyte cell surface marker expression in the tumor; and g) an increase in mRNA levels of INF-g related genes such as one or more or all of the genes of Tables 23 and 24.

### BRIEF DESCRIPTION OF THE DRAWINGS

FIG. 1 shows the plasmid map of a vector called pOMI-PIIM (OncoSec Medical Incorporated-Polycistronic IL-12 Immune Modulator) for the expression of both human IL-12 and a FLT3L-NYESO1 fusion protein.

FIG. 2 illustrates the activity of tissue culture cell-conditioned media containing secreted IL-12 p70 heterodimers expressed from pOMI-PIIM as measured using HEK Blue reporter cells. Controls (Addition of neutralizing anti-IL12 antibodies; conditioned media from un-transfected cells) are shown with dotted lines.

FIG. 3 illustrates the ability of intratumoral electroporation of pOMI-PIIM to control the growth of both primary (treated) and contralateral (untreated) B16-F10 tumors in mice (black line). Intratumoral electroporation of pUMVC3 (empty vector control) shown for comparison (dotted line).

FIG. 4 illustrates the ability of Flt3L fusion proteins produced from pOMI-PIIM to mature human dendritic cells in vitro. As compared with empty vector (EV) and inactive mutant Flt3L (H8R) controls, Flt3L-NY-ESO-1 significantly increased expression of A. CD80 and B. CD86 on primary human immature dendritic cells: *=p<0.05, **=p<0.01, ***=p<0.001.

FIG. 5 illustrates A. % TNF-α positive cells or B. % IFN-γ-positive cells following no treatment, NY-ESO-1(157-165) treatment, EV alone treatment, Flt3L-NY-ESO-1 treatment, or Flt3L-NY-ESO-1(H8R) treatment: *=p<0.05, **=p<0.01, ***=p<0.001.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

“Translation modulating element” or “translation modifier” as used herein, means a specific translation initiator or ribosomal skipping modulator wherein a picornavirus-derived sequence in the nascent polypeptide chain prevents covalent amide linkage with the next amino acid. Incorporation of this sequence results in co-expression of each chain of a heterodimeric protein with equal molar levels of the translated polypeptides. In some embodiments, the translation modifier is a 2A family of ribosomal skipping modulators. A 2A translation modified can be, but is not limited to, P2A, T2A, E2A and F2A, all of which share the PG/P cleavage site (See Table 5). In some embodiments, the translation modifier is an internal ribosomal entry sites (IRES).

In accordance with the present invention there may be employed conventional molecular biology, microbiology, and recombinant DNA techniques within the skill of the art. Such techniques are explained in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

The terms “nucleic acid”, “nucleotide sequence” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

A “polynucleotide sequence,” “nucleic acid sequence” or “nucleotide sequence” is a series of nucleotides in a nucleic acid, such as DNA or RNA, and means any chain of two or more nucleotides.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

A “coding sequence” or a sequence “encoding” an expression product such as a RNA or peptide (e.g., an immunoglobulin chain), is a nucleotide sequence that, when expressed, results in production of the product.

As used herein, the term “oligonucleotide” refers to a nucleic acid, generally of no more than about 300 nucleotides (e.g., 30, 40, 50, 60, 70, 80, 90, 150, 175, 200, 250 or 300), that may be hybridizable to a genomic DNA molecule, a cDNA molecule, or an mRNA molecule encoding a gene, mRNA, cDNA, or other nucleic acid of interest. Oligonucleotides are usually single-stranded, but may be double-stranded. Oligonucleotides can be labeled, e.g., by incorporation of 32P-nucleotides, 3H-nucleotides, 14C-nucleotides, 35S-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the gene, or to detect the presence of nucleic acids. Generally, oligonucleotides are prepared synthetically, e.g., on a nucleic acid synthesizer.

“Operable linkage” or being “operably linked” refers to the juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “plasmid” or “vector” includes any known delivery vector including a bacterial delivery vector, a viral vector delivery vector, a peptide immunotherapy delivery vector, a DNA immunotherapy delivery vector, an episomal plasmid, an integrative plasmid, or a phage vector. The term “vector” refers to a construct which is capable of delivering, and, optionally, expressing, one or more fusion polypeptides in a host cell. In some embodiments, the polynucleotide is the circular plasmid pOMIP2A or pOMI-PIIM.

A “protein sequence,” “peptide sequence” or “polypeptide sequence,” or “amino acid sequence” refers to a series of two or more amino acids in a protein, peptide or polypeptide.

The terms “protein,” “polypeptide,” and “peptide,” used interchangeably herein, refer to polymeric forms of amino acids of any length, including coded and non-coded amino acids and chemically or biochemically modified or derivatized amino acids. The terms include polymers that have been modified, such as polypeptides having modified peptide backbones.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The term “fusion protein” refers to a protein comprising two or more peptides linked together by peptide bonds or other chemical bonds. The peptides can be linked together directly by a peptide or other chemical bond. For example, a chimeric molecule can be recombinantly expressed as a single-chain fusion protein. Alternatively, the peptides can be linked together by a “linker” such as one or more amino acids or another suitable linker between the two or more peptides.

The term “isolated polynucleotide” or “isolated polypeptide” includes a polynucleotide (e.g., RNA or DNA molecule, or a mixed polymer) or a polypeptide, respectively, which is partially or fully separated from other components that are normally found in cells or in recombinant DNA expression systems or any other contaminant. These components include, but are not limited to, cell membranes, cell walls, ribosomes, polymerases, serum components and extraneous genomic sequences.

An isolated polynucleotide (e.g., pOMI-PIIM) or polypeptide will, preferably, be an essentially homogeneous composition of molecules but may contain some heterogeneity.

The term “host cell” includes any cell of any organism that is selected, modified, transfected, transformed, grown, or used or manipulated in any way, for the production of a substance by the cell, for example the expression or replication, by the cell, of a gene, a polynucleotide such as a circular plasmid (e.g., pOMI-PIIM) or RNA or a protein. For example, a host cell may be a mammalian cell or bacterial cell (e.g., E. coli) or any isolated cell capable of maintaining a described expression vector and promoting expression of a polypeptide encoded by expression vector.

Vectors, such as pOMI-PIIM, may be introduced into host cells according to any of the many techniques known in the art, e.g., dextran-mediated transfection, polybrene-mediated transfection, protoplast fusion, electroporation, calcium phosphate co-precipitation, lipofection, direct microinjection of the vector into nuclei, or any other means appropriate for a given host cell type.

A “cassette” or an “expression cassette” refers to a DNA coding sequence or segment of DNA that codes for an expression product (e.g., peptide or RNA) that can be inserted into a vector, e.g., at defined restriction sites. The expression cassette may comprise a promoter and/or a terminator and/or polyA signal operably linked to the DNA coding sequence.

In general, a “promoter” or “promoter sequence” is a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with or operably linked to other expression control sequences, including enhancer and repressor sequences or with a nucleic acid to be expressed. An expression control sequence is operably associated with or operably linked to a promoter if it regulates expression from said promoter.

The promoter used for gene expression in pOMI-PIIM is the human CMV immediate early promoter (Boshart et al., Cell 41:521-530 (1985); Foecking et al., Gene 45:101-105 (1986). The hCMV promoter provides a high level of expression in a variety of mammalian cell types.

A coding sequence is “under the control of”, “functionally associated with”, “operably linked to” or “operably associated with” transcriptional and translational control sequences in a cell when the sequences direct or regulate expression of the sequence. For example, a promoter operably linked to a gene will direct RNA polymerase mediated transcription of the coding sequence into RNA, preferably mRNA, which may then be spliced (if it contains introns) and, optionally, translated into a protein encoded by the coding sequence. A terminator/polyA signal operably linked to a gene terminates transcription of the gene into RNA and directs addition of a polyA signal onto the RNA.

The terms “express” and “expression” mean allowing or causing the information in a gene, RNA or DNA sequence to become manifest; for example, producing a protein by activating the cellular functions involved in transcription and translation of a corresponding gene. “Express” and “expression” include transcription of DNA to RNA and of RNA to protein. A DNA sequence is expressed in or by a cell to form an “expression product” such as an RNA (e.g., mRNA) or a protein. The expression product itself may also be said to be “expressed” by the cell.

The term “transformation” means the introduction of a nucleic acid into a cell. The introduced gene or sequence may be called a “clone”. A host cell that receives the introduced DNA or RNA has been “transformed” and is a “transformant” or a “clone.” The DNA or RNA introduced to a host cell can come from any source, including cells of the same genus or species as the host cell, or from cells of a different genus or species. Examples of transformation methods, which are very well known in the art, include liposome delivery, electroporation, CaPO4 transformation, DEAE-Dextran transformation, microinjection and viral infection.

Expression vectors, which comprise polynucleotides, are disclosed herein. The term “vector” may refer to a vehicle (e.g., a plasmid) by which a DNA or RNA sequence can be introduced into a host cell, so as to transform the host and, optionally, promote expression and/or replication of the introduced sequence.

The described polynucleotides may be expressed in an expression system. The term “expression system” means a host cell and compatible vector which, under suitable conditions, can express a protein or nucleic acid which is carried by the vector and introduced to the host cell. Common expression systems include E. coli host cells and plasmid vectors, insect host cells and baculovirus vectors, and mammalian host cells and vectors such as plasmids, cosmids, BACs, YACs and viruses such as adenovirus and adenovirus associated virus (AAV).

The terms “immunostimulatory cytokine” or “immunostimulatory cytokines” refer to protein naturally secreted by cells involved in immunity that have the capacity to stimulate an immune response.

The term “antigen” is used herein to refer to a substance that, when placed in contact with a subject or organism (e.g., when present in or when detected by the subject or organism), results in a detectable immune response from the subject or organism. An “antigenic peptide” refers to a peptide that leads to the mounting of an immune response in a subject or organism when present in or detected by the subject or organism. For example, such an “antigenic peptide” may encompass proteins that are loaded onto and presented on MI-IC class I and/or class II molecules on a host cell's surface and can be recognized or detected by an immune cell of the host, thereby leading to the mounting of an immune response against the protein. Such an immune response may also extend to other cells within the host, such as diseased cells (e.g., tumor or cancer cells) that express the same protein.

The phrase “genetic adjuvants containing shared tumor antigens” as used herein refers to targeting the Ag encoded by DNA through genetically fusing the Ag to molecules binding cell surface receptors as described in Table 1. Additional targeting components of genetic adjuvants are described in Table 2. Genetic adjuvants described here can act to accelerate, prolong, enhance or modify antigen-specific immune responses when used in combination with specific antigens.

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

### II. General

Described are expression vectors that allow adequate expression of multiple proteins following transfection of an in vivo cell, particularly a tumor cell or other cells, e.g., an immune cell, in the tumor microenvironment.

Vectors are provided that contain some or all of the modifications described herein designed to improve their efficacy and safety. The optimization of the vectors includes the incorporation of sequences encoding appropriate peptides and the tailoring of sites to improve gene expression. A peptide is understood to be any translation product regardless of size, and whether or not post-translationally modified, as, for example, in glycosylation and phosphorylation.

Described are expression vectors comprising the translation control element, e.g., P2A, operatively linked to gene sequences to be expressed. In certain embodiments, the expression vector comprises at least two nucleic acid sequences to be translated and the translation control element is operatively linked to at least one of the sequences to be translated. Vectors are known or can be constructed by those skilled in the art and contain all expression elements necessary to achieve the desired transcription of the sequences in addition to the sequence described herein as shown in the Examples herein below. The vectors contain elements for use in either prokaryotic or eukaryotic host systems depending on their use. One of ordinary skill in the art will know which host systems are compatible with a particular vector.

Recombinant gene expression depends upon transcription of the appropriate gene and efficient translation of the message. A failure to perform correctly either one of these processes can result in the failure of a given gene to be expressed. This is further complicated when more than one gene needs to be expressed from a single plasmid. Traditionally, internal ribosomal entry sites (IRES's) were used between the genes to be expressed. IRES's have limitations because of their size and the translation efficiency of the second gene is much lower than the first. Recent studies have found that the use of picornavirus polyprotein 2A (“P2A”) peptide results in expression of multiple proteins flanking the P2A peptide with 1-to-1 stoichiometry (see, e.g., Kim et al (2011) PloS One 6:318556).

In some embodiments, expression vectors for expression of diverse immunomodulators including, e.g., heterodimeric proteins such as IL-12 (GenBank reference # s NP_000873.2, NP_002178.2) and genetic adjuvants, e.g. FLT3 ligand extracellular domain (FLT3L, GenBank # XM_017026533.1) containing shared tumor antigens, e.g., FLT3L-NYESO1 fusion protein, are described. In some embodiments, the expression vectors are delivered to a tumor (intratumoral delivery) via in vivo electroporation.

TABLE 1
Genetic Adjuvants fused to shared tumor antigens or viral antigens (Flt3L protein fusions)

| Gene | Structure | Reference |
|---|---|---|
| NY-ESO-1 | Fusion of full length protein to ECD of | Gnjatic et al., Advances in Cancer Res. |
| | FLT3L | 2006 |
| NY-ESO-1 | Fusion of amino acid# 80-180 to ECD of | Sabado-RL, Cancer Immunol Res 2015 |
| | FLT3L | MARCH; 3(3) |
| NY-ESO-1 | Fusion of overlapping peptides: | |
| | Amino acid# 81-100, 87-111, 157- | |
| | 165, 157-170, 161-180 to ECD of FLT3L | |
| NY-ESO-1 | Fusion of amino acid # 157-165 to ECD | RAPOPORT-AP, NATURE |
| | of FLT3L | MEDICINE, 2015 AUGUST 21(8) |
| MAGE-A1 | Fusion of full length protein or antigenic | Almeida et al., Nuc, Acids Res 2009; |
| | peptides to ECD of FLT3L | CTDatabase, Ludwig Institute for Cancer |
| | | Research |
| MAGE-A2 | Fusion of full length protein or antigenic | Almeida et al., Nuc, Acids Res 2009; |
| | peptides to ECD of FLT3L | CTDatabase, Ludwig Institute for Cancer |
| | | Research |
| MAGE-A3 | Fusion of full length protein or antigenic | Almeida et al., Nuc, Acids Res 2009; |
| | peptides to ECD of FLT3L | CTDatabase, Ludwig Institute for Cancer |
| | | Research |
| MAGE-A10 | Fusion of full length protein or antigenic | Almeida et al., Nuc, Acids Res 2009; |
| | peptides to ECD of FLT3L | CTDatabase, Ludwig Institute for Cancer |
| | | Research |
| SSX-2 | Fusion of full length protein or antigenic | Almeida et al., Nuc, Acids Res 2009; |
| | peptides to ECD of FLT3L | CTDatabase, Ludwig Institute for Cancer |
| | | Research |
| MART-1 | Fusion of full length protein or antigenic | Li et al., J. Immunol. 2010, 184: 452 |
| | peptide ELAGIGILTV to ECD of | |
| | FLT3L | |
| Tyrosinase | Fusion of antigenic peptide | Skipper et al., J. Exp. Med 1996, |
| | YMDGTMSQV to ECD of FLT3L | 183: 527 |
| Gp100 | Fusion of full length protein or antigenic | Bakker et al., J. Exp. Med. 1994, |
| | peptides to ECD of FLT3L | 179: 1005 |
| Survivin | Fusion of full length protein or antigenic | Schmidt et al., Blood 2002, 102:571 |
| | peptide ELTLGEFLKL to ECD of | |
| | FLT3L | |
| hTERT | Fusion of full length protein or antigenic | Vonderheide et al., Nature 2002, 21:674 |
| | peptides to ECD of FLT3L | |
| WT1 | Fusion of full length protein or antigenic | Cheever et al., Clin. Cancer Res. 2009, |
| | peptides to ECD of FLT3L | 15: 5323 |
| PSMA | Fusion of full length protein or antigenic | Chudley et al., Cancer Immunol |
| | peptides to ECD of FLT3L | Immunother. 2012, 61: 2161 |
| PRS pan-DR | Fusion of full length protein or antigenic | Almeida et al., Nuc, Acids Res 2009; |
| | peptides to ECD of FLT3L | CTDatabase, Ludwig Institute for Cancer |
| | | Research |
| B7-H6 | Full length protein or fusion of full | Brandt et al., J. Exp Med. 2009, |
| | length protein to ECD of FLT3L | 206:1495 |
| HPV E7 | Full length protein or fusion of full | Huang et al., Cancer Res. 2001 61:1080; |
| | length protein to ECD of FLT3L | Seo et al., Vaccine 2009 27:5906; Lin et |
| | | al., |
| HPV16 E6/E7 | 1-85 aa E6, 1-65 aa E7, 71-158 aa E6, | Kim et al, Nature 2014 5: 5317 |
| | 51-98 aa E7 fused to ECD of FLT3L | |
| HPV16 E6/E7 | E6 mutant L50A; E6 mutant ETNL146- | Wieking et al., 2012, Cancer Gene Ther. |
| | 151AAAA; E7 mutant H2P; E7 mutant | 19: 667 |
| | C24G; E7 mutant E46A; E7 mutant | |
| | L67R | |
| HPV11 E6 | 44-51 aa E6 | Peng et al., 2010, Larynoscope 120: 504 |
| HPV6b/11 E7 | 21-29 aa E7, 82-90 aa E7 | Peng et al., 2016, Cancer Immunol. |
| | | Immunother. 65: 261 |
| HCV-NS3 | Fusion of full length protein or antigenic | Grubor-Bauk et al., 2016, Gene Ther. |
| | peptides fused to ECD of FLT3L | 23: 26 |
| Influenza HA | Fusion of full length protein or antigenic | Chow et al., 1979. Infect Immun. 25: 103 |
| and NA | peptides to ECD of FLT3L | |
| Polyoma-virus | MCPyV LTA aa1-258, aa136-160; | Zeng et al., Vaccine 2012 30: 1322; |
| | various other peptides from VP1, LTA, | Lyngaa et al., 2014, Clin Can Res 2014, |
| | and STA | 20: 1768 |

Additional genetic adjuvants are also contemplated (Table 2).

TABLE 2
Genetic Adjuvants

| Gene | Structure | Reference |
|---|---|---|
| Flt3 ligand | Extracellular | XM_017026533.1 |
| | domain (ECD) | |
| LAMP-1 | | XM_011537494.1 |
| Calreticulin | Full length protein | NM_004343; Cheng et al., |
| | | 2001, J Clin Invest. |
| | | 108: 669 |
| Human heat shock | Full length protein | Rivoltini et al., 2003. |
| protein 96 | | J. Immunol. 171: 3467 |
| GM-CSF | Full length protein | NM_000758.3 |
| CSF Receptor 1 | | NM_001288705.2 |

In some embodiments, we describe expression vectors comprising the nucleotide sequence of SEQ ID NO: 8 or a nucleotide sequence having at least 70% identity to the nucleotide sequence of SEQ ID NO: 8. In some embodiments, an expression vector comprises a sequence having greater than 70%, 72%, 75%, 78%, 80%, 82%, 83%, 85%, 87%, 88%, 90%, 92%, 93%, 95%, 96%, 97%, 98%, or 99% identity to the nucleotide sequence of SEQ ID NO: 8. In some embodiments, the nucleotide sequence of SEQ ID NO: 8 or the nucleotide sequence having at least 70% identity to the nucleotide sequence of SEQ ID NO: 8 is operably linked to a CMV promoter.

In some embodiments, we describe expression vectors encoding a polypeptide comprising the amino acid sequence of SEQ ID NO: 9 or a polypeptide having at least 70% identity to the amino acid sequence of SEQ ID NO: 9. In some embodiments, an expression vector encodes a polypeptide comprising an amino acid sequence having greater than 70%, 72%, 75%, 78%, 80%, 82%, 83%, 85%, 87%, 88%, 90%, 92%, 93%, 95%, 96%, 97%, 98%, or 99% identity to the amino acid sequence of SEQ ID NO: 9. In some embodiments, the expression vector encodes a polypeptide having at least 80%, at least 85%, and least 90%, at least 95%, at least 97%, or at least 99% homology to the amino acid sequence of SEQ ID NO: 9.

In some embodiments, we describe expression vectors comprising the nucleotide sequence of SEQ ID NO: 10 or a nucleotide sequence having at least 70% identity to the nucleotide sequence of SEQ ID NO: 10. In some embodiments, an expression vector comprises a sequence having greater than 70%, 72%, 75%, 78%, 80%, 82%, 83%, 85%, 87%, 88%, 90%, 92%, 93%, 95%, 96%, 97%, 98%, or 99% identity to the nucleotide sequence of SEQ ID NO: 10. In some embodiments, the nucleotide sequence of SEQ ID NO: 10 or the nucleotide sequence having at least 70% identity to the nucleotide sequence of SEQ ID NO: 10 is operably linked to a CMV promoter.

In some embodiments, we describe expression vectors encoding a polypeptide comprising the amino acid sequence of SEQ ID NO: 11 or a polypeptide having at least 70% identity to the amino acid sequence of SEQ ID NO: 11. In some embodiments, an expression vector encodes a polypeptide comprising an amino acid sequence having greater than 70%, 72%, 75%, 78%, 80%, 82%, 83%, 85%, 87%, 88%, 90%, 92%, 93%, 95%, 96%, 97%, 98%, or 99% identity to the amino acid sequence of SEQ ID NO:11. In some embodiments, the expression vector encodes a polypeptide having at least 80%, at least 85%, and least 90%, at least 95%, at least 97%, or at least 99% homology to the amino acid sequence of SEQ ID NO: 11.

In some embodiments, we describe expression vectors comprising the nucleotide sequence of SEQ ID NO: 12 or a nucleotide sequence having at least 70% identity to the nucleotide sequence of SEQ ID NO: 12. In some embodiments, an expression vector comprises a sequence having greater than 70%, 72%, 75%, 78%, 80%, 82%, 83%, 85%, 87%, 88%, 90%, 92%, 93%, 95%, 96%, 97%, 98%, or 99% identity to the nucleotide sequence of SEQ ID NO: 12.

In some embodiments, we describe expression vectors comprising the nucleotide sequence of SEQ ID NO. 1 or a nucleotide sequence having at least 70% identity to the nucleotide sequence of SEQ ID NO: 1 In some embodiments, an expression vector comprises, consists essentially of, or consists of a sequence having greater than 70%, 72%, 75%, 78%, 80%, 82%, 83%, 85%, 87%, 88%, 90%, 92%, 93%, 95%, 96%, 97%, 98%, or 99% identity to the nucleotide sequence of SEQ ID NO: 1.

### III. Devices and Uses

In some embodiments, the described expression vectors are delivered by intratumoral gene electrotransfer. The described expression vectors can be used to generate adequate concentrations of several recombinantly expressed immunomodulatory molecules such as, multimeric cytokines or combination of multimeric cytokines, co-stimulatory molecules in native or engineered forms, genetic adjuvants containing shared tumor antigens, etc. To achieve transfer of the expression vectors into a tissue, e.g., a tumor, an electroporation device can be employed.

The devices and methods of the present embodiment work to treat cancerous tumors by delivering electrical therapy continuously and/or in pulses for a period of time ranging from a fraction of a second to several days, weeks, and/or months to tumors. In a preferred embodiment, electrical therapy is direct current electrical therapy.

The term “electroporation” (i.e. rendering cellular membranes permeable) as used herein may be caused by any amount of coulombs, voltage, and/or current delivered to a patient in any period of time sufficient to open holes in cellular membranes (e.g. to allow diffusion of molecules such as pharmaceuticals, solutions, genes, and other agents into a viable cell).

Delivering electrical therapy to tissue causes a series of biological and electrochemical reactions. At a high enough voltage, cellular structures and cellular metabolism are severely disturbed by the application of electrical therapy. Although both cancerous and non-cancerous cells are destroyed at certain levels of electrical therapy tumor cells are more sensitive to changes in their microenvironment than are non-cancerous cells. Distributions of macroelements and microelements are changed as a result of electrical therapy. Destruction of cells in the vicinity of the electroporation is known as irreversible electroporation.

The use of reversible electroporation is also contemplated. Reversible electroporation occurs when the electricity applied with the electrodes is below the electric field threshold of the target tissue. Because the electricity applied is below the cells' threshold, cells are able to repair their phospholipid bilayer and continue on with their normal cell functions. Reversible electroporation is typically done with treatments that involve getting a drug or gene (or other molecule that is not normally permeable to the cell membrane) into the cell. (Garcia, et al. (2010) “Non-thermal irreversible electroporation for deep intracranial disorders”. 2010 Annual International Conference of the IEEE Engineering in Medicine and Biology: 2743-6.)

In a single electrode configuration, voltage may be applied for fractions of seconds to hours between a lead electrode and the generator housing, to begin destruction of cancerous tissue. Application of a given voltage may be in a series of pulses, with each pulse lasting fractions of a second to several minutes. In certain embodiments, the pulse duration or width can be from about 10 μs to about 100 ms. Low voltage may also be applied for of a duration of fractions of seconds to minutes, which may attract white blood cells to the tumor site. In this way, the cell-mediated immune system may remove dead tumor cells and may develop antibodies against tumor cells. Furthermore, the stimulated immune system may attack borderline tumor cells and metastases.

Various adjuvants may be used to increase any immunological response, depending on the host species, including but not limited to Freund's adjuvant (complete and incomplete), mineral salts such as aluminum hydroxide or aluminum phosphate, various cytokines, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and or coupling with molecules such as keyhole limpet hemocyanin, tetanus toxoid, diphtheria toxoid, ovalbumin, cholera toxin or fragments thereof.

U.S. Pat. No. 7,245,963 by Draghia-Akli, et al. describes modular electrode systems and their use for facilitating the introduction of a biomolecule into cells of a selected tissue in a body or plant. The modular electrode systems comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 describes an electroporation device, which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk (see, e.g., U.S. Patent Pub. 2005/0052630) is hereby incorporated by reference.

The electrode arrays and methods described in U.S. Pat. No. 7,245,963 and U.S. Patent Pub. 2005/0052630 are adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes.

Also encompassed are electroporation devices incorporating electrochemical impedance spectroscopy (“EIS”). Such devices provide real-time information on in vivo, in particular, intratumoral electroporation efficiency, allowing for the optimization of conditions. Examples of electroporation devices incorporating EIS can be found, e.g., in WO2016161201, which is hereby incorporated by reference.

Other alternative electroporation technologies are also contemplated. In vivo plasmid delivery can also be performed using cold plasma. Plasma is one of the four fundamental states of matter, the others being solid, liquid, and gas. Plasma is an electrically neutral medium of unbound positive and negative particles (i.e. the overall charge of a plasma is roughly zero). A plasma can be created by heating a gas or subjecting it to a strong electromagnetic field, applied with a laser or microwave generator. This decreases or increases the number of electrons, creating positive or negative charged particles called ions (Luo, et al. (1998) Phys. Plasma 5:2868-2870) and is accompanied by the dissociation of molecular bonds, if present.

Cold plasmas (i.e., non-thermal plasmas) are produced by the delivery of pulsed high voltage signals to a suitable electrode. Cold plasma devices may take the form of a gas jet device or a dielectric barrier discharge (DBD) device. Cold temperature plasmas have attracted a great deal of enthusiasm and interest by virtue of their provision of plasmas at relatively low gas temperatures. The provision of plasmas at such a temperature is of interest to a variety of applications, including wound healing, anti-bacterial processes, various other medical therapies and sterilization. As noted earlier, cold plasmas (i.e., non-thermal plasmas) are produced by the delivery of pulsed high voltage signals to a suitable electrode. Cold plasma devices may take the form of a gas jet device, a dielectric barrier discharge (DBD) device or multi-frequency harmonic-rich power supply.

Dielectric barrier discharge device, relies on a different process to generate the cold plasma. A dielectric barrier discharge (DBD) device contains at least one conductive electrode covered by a dielectric layer. The electrical return path is formed by the ground that can be provided by the target substrate undergoing the cold plasma treatment or by providing an in-built ground for the electrode. Energy for the dielectric barrier discharge device can be provided by a high voltage power supply, such as that mentioned above. More generally, energy is input to the dielectric barrier discharge device in the form of pulsed DC electrical voltage to form the plasma discharge. By virtue of the dielectric layer, the discharge is separated from the conductive electrode and electrode etching and gas heating is reduced. The pulsed DC electrical voltage can be varied in amplitude and frequency to achieve varying regimes of operation. Any device incorporating such a principle of cold plasma generation (e.g., a DBD electrode device) falls within the scope of various described embodiments.

Cold plasma has been employed to transfect cells with foreign nucleic acids. In particular, transfection of tumor cells (see, e.g., Connolly, et al. (2012) Human Vaccines & Immunotherapeutics 8:1729-1733; and Connolly et al (2015) Bioelectrochemistry 103: 15-21).

The devices are contemplated for use in patients afflicted with cancer or other non-cancerous (benign) growths. These growths may manifest themselves as any of a lesion, polyp, neoplasm (e.g. papillary urothelial neoplasm), papilloma, malignancy, tumor (e.g. Klatskin tumor, hilar tumor, noninvasive papillary urothelial tumor, germ cell tumor, Ewing's tumor, Askin's tumor, primitive neuroectodermal tumor, Leydig cell tumor, Wilms' tumor, Sertoli cell tumor), sarcoma, carcinoma (e.g. squamous cell carcinoma, cloacogenic carcinoma, adenocarcinoma, adenosquamous carcinoma, cholangiocarcinoma, hepatocellular carcinoma, invasive papillary urothelial carcinoma, flat urothelial carcinoma), lump, or any other type of cancerous or non-cancerous growth. Tumors treated with the devices and methods of the present embodiment may be any of noninvasive, invasive, superficial, papillary, flat, metastatic, localized, unicentric, multicentric, low grade, and high grade.

The devices are contemplated for use in numerous types of malignant tumors (i.e. cancer) and benign tumors. For example, the devices and methods described herein are contemplated for use in adrenal cortical cancer, anal cancer, bile duct cancer (e.g. periphilar cancer, distal bile duct cancer, intrahepatic bile duct cancer) bladder cancer, benign and cancerous bone cancer (e.g. osteoma, osteoid osteoma, osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia, triple negative breast cancer (TNBC)), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adenocarcinoma, clear cell) esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Cutaneous T-Cell Lymphoma (CTCL), Kaposi's sarcoma, kidney cancer (e.g. renal cell cancer), liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, squamous cell carcinomas of the head and neck (including, but not limited to nasal cavity and paranasal sinus cancer (e.g. esthesioneuroblastoma, midline granuloma), salivary gland cancer, nasopharyngeal cancer, neuroblastoma, laryngeal and hypopharyngeal cancer, oral cavity cancers, and oropharyngeal cancer), ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), skin cancer, both melanoma and non-melanoma skin cancer (including Merkel Cell Carcinoma), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma).

### IV. Intratumoral Electroporation Parameters

Typically, the electric fields needed for in vivo cell electroporation, in particular, intratumoral electroporation (IT-EP), are generally similar in magnitude to the fields required for cells in vitro. In one embodiment, the magnitude of the electric field range from approximately, 10 V/cm to about 1500 V/cm, from about 200 V/cm to 1500 V/cm, from about 200 V/cm to 800 V/cm, from about 200 V/cm to 500 V/cm. In one embodiment the field strength is about 200 V/cm to about 400 V/cm, and preferably from about 400 V/cm.

The pulse length or frequency can be about 10 μs to about 100 ms, about 100 μs to about 50 ms, about 500 μs to 10 ms. In one embodiment the field strength is about 400 V/cm and the pulse length is about 10 ms. There can be any desired number of pulses, typically one to 100 pulses per second. The interval between pulses sets can be any desired time, such as one second. The waveform, electric field strength and pulse duration may also depend upon the type of cells and the type of molecules that are to enter the cells via electroporation.

The plasmid encoded immunostimulatory cytokine is delivered by electroporation at least one, two, or three days of each cycle or alternating cycles. In certain embodiments, the cytokine is delivered on days 1, 5, and 8 of each cycle. In a preferred embodiment, the cytokine is delivered on days 1, 3, and 8 of every odd numbered cycle. In certain embodiment if the plasmid contains P2A translation elements, the plasmid-encoded cytokine is delivered as a single treatment on day 1 only.

The P2A containing plasmid encoding the immunostimulatory cytokine is dosed at about 1 μg to 100 μg, about 10 μg to about 50 μg, about 10 μg to about 25 μg. In an alternative embodiment, the amount of plasmid is determine by calculation of target tumor volume, and administering ¼ of this volume of 0.5 mg/ml solution of the P2A containing plasmids.

### IV. Combination Therapies

The present disclosure encompasses methods of treating cancer in a human subject, the methods comprising the step(s) of administering to the subject a therapeutically effective amount one or more of the described expression vectors. In some embodiments, the described expression vector is administered in combination with electroporation.

In some embodiments, any of the described therapies is combined with one or more additional (i.e., second) therapeutics or treatments. The expression vector and additional therapeutics can be administered in a single composition or they made be administered separately. Non-limited examples of additional therapeutics include, but are not limited to, anti-cancer drug, anti-cancer biologic, antibody, anti-PD-1 inhibitor, anti-CTLA4 antagonist Ab, tumor vaccine, or other therapies known in the art.

It is contemplated that intratumoral electroporation (IT-EP) of DNA encoding immunomodulatory proteins can be administered with other therapeutic entities. Table 3 provides possible combinations. Administration of the combination therapies can be achieved by electroporation alone or a combination of electroporation and systemic delivery.

TABLE 3
Combination Therapies

| | Proposed delivery | |
|---|---|---|
| Combination | method | Reference |
| IT-pOMI-PIIM-EP + Anti-PD1 | Intratumoral Electroporation (“IT- | i.e. Quetglas et al. Can, Immol, |
| | EP”) of plasmids encoding | |
| antagonist Ab | cytokines, co-stimulators, | Res. 2015, 3: 449; Chen and |
| | immune-directors in pOMI-PIIM | Daud, Oncology 2016, 30: 442 |
| | plus systemic anti-PD-1 Ab | |
| | treatment | |

| | |
|---|---|
| 1. | co-administration |
| 2. | Administration of IT-EP, |

followed by systemic anti-
PD-1 inhibitor

| | |
|---|---|
| IT-pOMI-PIIM-EP + anti- | IT-EP of pOMI-PIIM plus |
| PDL1 antagonist Ab | systemic anti-PDL-1 Ab treatment |

| | |
|---|---|
| 1. | co-administration |
| 2. | sequential administration |

of IT-EP, followed by
systemic anti-PDL-1
inhibitor

| | | |
|---|---|---|
| IT-pOMI-PIIM-EP + CTLA4 | IT-EP of pOMI-PIIM plus | Vom Berg et al., 2013, J. Exp. |
| agonist antibody (“Ab”) or | systemic delivery of CTLA4 | Med. 210: 2803 |
| ligand | antagonist Abs | |

| | 1. | co-administration | |
|---|---|---|---|
| | 2. | sequential administration | |
| | | of IT-EP, followed by | |
| | | systemic anti-CTLA4 | |
| | | antagonist Ab. | |
| IT-pOMI-PIIM-EP + tumor | 1. | IT-EP of pOMI-PIIM + | Vergati et al., 2010. J. Biomed. |
| vaccine | | cytotoxic agent | Biotechnol. 2010: Article ID |
| (separately) to create local | | 596432 | |
| tumor antigen pool | | | |
| | 2. | IT-EP of pOMI-PIIM + | |
| | | system delivery of tumor | |
| | | vaccine (i.e. gp100 | |
| | | peptide vaccine for | |
| | | melanoma) | |
| IT-pOMI-PIIM-EP + | 1. | IT-EP of drug + pOMI- | i.e. Zhang et al., 2015, J. |
| Bleomycin, Gemzar, Cytozan, | | PIIM | Immunother. 38: 137 |
| 5-fluoro-uracil, Adriamycin or | 2. | IT-EP of pOMIP2A + | |
| other chemotherapeutic agent | | system delivery of drug | |
| IT-pOMI-PIIM-EP + small | 1. | IT-EP of pOMI-PIIM | Hu-Lieskovan et al., (2014) J. |
| molecule inhibitors (i.e. | | combined with local drug | Clin. Oncol. 32(21): 2248-54 |
| Sunitinib, Imatinib, | | delivery | |
| Vemurafenib, Trastuzumab, | 2. | IT-EP of pOMI-PIIM | Vanneman and Dranoff (2014) |

| | | |
|---|---|---|
| Bevacizumab, Cetuximb, | combined with systemic | Nat. Rev. Cancer 12(4): 237- |
| rapamycin, Bortezomib, | drug treatment | 251 |
| PI3K-AKT inhibitors, IAP | | |
| inhibitors | | |

| | | |
|---|---|---|
| IT-pOMI-PIIM-EP + targeted | Sublethal radiation dose locally at | Almo SC, Guha C. (2014) |
| radiation | tumor site, followed by IT-EP of | Radiation Res. 182(2): 230-238. |
| | pOMI-PIIM | |

The described expression vectors and/or compositions can be used in methods for therapeutic treatment of cancer. The cancer can be, but is not limited to: melanoma, breast cancer, triple negative breast cancer, Merkel Cell Carcinoma, CTCL, head and neck squamous cell carcinoma or other cancer as described above. Such methods comprise administration of an expression vector by electroporation.

In some embodiments, at least one of the described expression vectors is used in the preparation of a pharmaceutical composition (i.e., medicament) for treatment of a subject that would benefit expression of IL12 and FLT3L-NY-ESO in a tumor. In some embodiments, the described pharmaceutical compositions are used to treat cancer in a subject.

As used herein, a pharmaceutical composition or medicament comprises a pharmacologically effective amount of at least one of the described expression vectors. In some embodiments, a pharmaceutical composition or medicament further comprises one or more pharmaceutically acceptable excipients. Pharmaceutically acceptable excipients (excipients) are substances other than the Active Pharmaceutical ingredient (API, therapeutic product, e.g., expression vector) that have been appropriately evaluated for safety and are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, binders, buffering agents, carriers, coating agents, colors, delivery enhancers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells that express or comprise the herein described expression vectors may be used as “pharmaceutical compositions”. As used herein, “pharmacologically effective amount,” “therapeutically effective amount,” or simply “effective amount” refers to that amount of an expression vector to produce the intended pharmacological, therapeutic or preventive result.

In some embodiments, a described expression vector can be used to: lower mean tumor volume in a treated tumor lesion, lower mean tumor volume in an untreated contralateral tumor lesion, induce an influx of lymphocytes into the tumor, induce an increase of circulating tumor-specific CD8+ T cells, increase lymphocyte and monocyte cell surface marker expression in the tumor, and/or increase mRNA levels of any of the INF-γ related genes of Tables 23 and 24.

In some embodiments, intratumoral expression of IL-12 is increased by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject prior to being administered the expression vector or to a subject not receiving the expression vector. In some embodiments intratumoral expression of IL-12 is increased by at least 1×, at least 2×, at least 3×, at least 3.6×, at least 4×, or at least 5× relative to the subject prior to being administered the expression vector or to a subject not receiving the expression vector.

In some embodiments, mean tumor volume in a treated tumor lesion is reduced by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject prior to being administered the expression vector or to a subject not receiving the expression vector.

In some embodiments, mean tumor volume in an untreated contralateral tumor lesion is reduced by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject prior to being administered the expression vector or to a subject not receiving the expression vector.

In some embodiments, influx of lymphocytes into the tumor is increase by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject prior to being administered the expression vector or to a subject not receiving the expression vector. In some embodiments, influx of lymphocytes into the tumor is increased by at least 1×, at least 2×, at least 3×, at least 4×, or at least 5× relative to the subject prior to being administered the expression vector or to a subject not receiving the expression vector.

In some embodiments, circulating tumor-specific CD8+ T cells in the subject are increased by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject prior to being administered the expression vector or to a subject not receiving the expression vector. In some embodiments, circulating tumor-specific CD8+ T cells in the subject are increased by at least 1×, at least 2×, at least 3×, at least 4×, or at least 5× relative to the subject prior to being administered the expression vector or to a subject not receiving the expression vector.

In some embodiments, lymphocyte and monocyte cell surface marker expression in the tumor is increased by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject prior to being administered the expression vector or to a subject not receiving the expression vector. In some embodiments, lymphocyte and monocyte cell surface marker expression in the tumor is increased by at least 1×, at least 2×, at least 3×, at least 4×, or at least 5× relative to the subject prior to being administered the expression vector or to a subject not receiving the expression vector.

In some embodiments, mRNA levels of any of the INF-γ related genes of Tables 23 and 24 in the tumor is increased by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject prior to being administered the expression vector or to a subject not receiving the expression vector. In some embodiments, mRNA levels of any of the INF-γ related genes of Tables 23 and 24 in the tumor is increased by at least 1×, at least 2×, at least 3×, or at least 5× relative to the subject prior to being administered the expression vector or to a subject not receiving the expression vector.

In some embodiments, the described expression vectors or compositions containing the expression vectors can be delivered to a tumor or tumor lesion by electroporation. In general, any suitable electroporation method recognized in the art for delivering a nucleic acid molecule (in vitro or in vivo) can be adapted for use with the described expression vectors.

The described expression vectors and pharmaceutical compositions comprising the expression vectors disclosed herein may be packaged or included in a kit, container, pack, or dispenser. The expression vectors and pharmaceutical compositions comprising expression vectors may be packaged in pre-filled syringes or vials. A kit can comprise a reagent utilized in performing a method disclosed herein. A kit can also comprise a composition, tool, or instrument disclosed herein. For example, such kits can comprise any of the described expression vectors. In some embodiments, the kit comprises one or more the described expression vectors and an electroporation device. In some embodiments, the kit comprises one or more the described expression vectors and one or more electrode disks, needle electrodes, and injection needles. Although model kits are described below, the contents of other useful kits will be apparent in light of the present disclosure.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect as described herein can be used in combination with any other unless specifically indicated otherwise. Although the embodiments are described in some detail by way of illustration and example for purposes of clarity and understanding, it will be apparent that certain changes and modifications may be practiced within the scope of the appended claims.

### Listing of Embodiments

The subject matter disclosed herein includes, but is not limited to, the following embodiments.

1. An expression vector comprising the nucleic acid sequence of SEQ ID NO: 1.

2. An expression vector comprising a nucleic acid encoding a polypeptide comprising an amino acid having at least 70% identity to the amino acid sequence of SEQ ID NO: 9.

3. The expression vector of embodiment 2, wherein the polypeptide comprises the amino acid sequence of SEQ ID NO: 9.

4. The expression vector of embodiment 2 or 3, wherein the nucleic acid comprises a nucleotide sequence having at least 70% identity to the nucleotide sequence of SEQ ID NO: 8

5. The expression vector of embodiment 4, wherein the nucleic acid comprises the nucleotide sequence of SEQ ID NO: 8.

6. The expression vector of embodiment 4 or 5, wherein the nucleic acid is operably linked to a nucleic acid encoding a P2A translation modification element and a nucleic acid encoding a FLT-3L peptide fused to at least one antigen.

7. The expression vector of embodiment 6, wherein the antigen is selected from the group consisting of: NYESO-1, OVA, RNEU, MAGE-A1, MAGE-A2, Mage-A10, SSX-2, Melan-A, MART-1, Tyr, Gp100, LAGE-1, Survivin, PRS pan-DR, CEA peptide CAP-1, OVA, HCV-NS3, TERT, WT1, PSMA, and an HPV vaccine peptide.

8. The expression vector of embodiment 7, wherein the antigen is NYESO-1.

9. The expression vector of any one of embodiments 2-8, wherein the nucleic acid is operably linked to a CMV promoter.

10. The expression vector of any one of embodiments 2-9, wherein the polypeptide comprises an amino acid sequence having at least 70% identity to the amino acid sequence of SEQ ID NO: 11.

11. The expression vector of embodiment 10, wherein the polypeptide comprises the amino acid sequence of SEQ ID NO: 11.

12. The expression vector of embodiment 10 or 11, wherein the nucleic acid comprises a nucleotide sequence having at least 70% identity to the nucleotide sequence of SEQ ID NO: 10.

13. The expression vector of embodiment 12, wherein the nucleic acid comprises the nucleotide sequence of SEQ ID NO: 10.

14. The expression vector of embodiment 12 or 13, wherein the nucleic acid is operably linked to a CMV promoter.

15. The expression vector of embodiment 14, wherein the expression vector comprises a nucleotide sequence having at least 70% identity to the nucleotide sequence of SEQ ID NO: 12.

16. The expression vector of embodiment 15, wherein the expression vector comprises the nucleotide sequence of SEQ ID NO: 12.

17. A method of treating a tumor in a subject, comprising delivering the expression vector any one of embodiments 1-16 into the tumor using at least one intratumoral electroporation pulse.

18. The method of embodiment 17, wherein the intratumoral electroporation pulse has a field strength of about 200 V/cm to about 1500 V/cm.

19. The method of embodiment 17 or 18, wherein the subject is a human.

20. The method of any one of embodiments 17-19, wherein the tumor is selected from the group of melanoma, triple negative breast cancer, Merkel Cell Carcinoma, CTCL, and head and neck squamous cell carcinoma.

21. The method of any one of embodiments 17-20, wherein the electroporation pulse is delivered by a generator capable of electrochemical impedance spectroscopy.

22. A method of treating a tumor in a subject, comprising administering at least one low voltage intratumoral electroporation (IT-EP) treatment that delivers an expression vector comprising:

a. the nucleotide sequence of SEQ ID NO: 1, SEQ ID NO: 8, SEQ ID NO: 10, or SEQ ID NO: 12;

b. a nucleotide sequence having at least 70% identity to the nucleotide sequence of SEQ ID NO: 1, SEQ ID NO: 8, SEQ ID NO: 10, or SEQ ID NO: 12;

c. a nucleotide sequence encoding a polypeptide comprising the amino acid sequence of SEQ ID NO: 9 or SEQ ID NO: 11; or

d. a nucleotide sequence encoding a polypeptide having at least 70% identity to the amino acid sequence of SEQ ID NO: 9 or SEQ ID NO: 11.

23. The method of embodiment 22, wherein the IT-EP treatment comprises a field strength from about 200 V/cm to about 500 V/cm and a pulse length of about 100 μs to about 50 ms.

24. The method of embodiment 23, wherein the treatment is one IT-EP treatment and comprises a field strength of about 350-450 V/cm and a pulse length of about 10 ms.

25. The method of embodiment 24, wherein the treatment is one IT-EP treatment and comprises a field strength of about 400 V/cm and a pulse length of about 10 ms.

26. The method of any one of embodiments 17-25, wherein the treatment comprises 1-10 10 ms electroporation pulses.

27. The method of embodiment 26, wherein the treatment comprises 5-10 10 ms electroporation pulses.

28. The method of embodiment 27, wherein the treatment comprises 8 10 ms electroporation pulses.

29. The method of any one of embodiments 22-28, wherein the treatment results in one or more or all of the following when compared to low voltage IT-EP treatment with an IL-12 encoding plasmid containing an IRES motif:

a. at least 3.6 times higher intratumoral expression of IL-12;

b. a lower mean tumor volume in a treated tumor lesion;

c. a lower mean tumor volume in an untreated contralateral tumor lesion;

d. a higher influx of lymphocytes into the tumor;

e. an increase of circulating tumor-specific CD8+ T cells;

f. an increase of lymphocyte and monocyte cell surface marker expression in the tumor; and

g. an increase in mRNA levels of INF-g related genes of Tables 23 and 24.

30. The expression vector of any of embodiments 1-16 for use in treating a tumor in a subject wherein treating comprises delivering the expression vector into the tumor using at least one intratumoral electroporation pulse.

31. The expression vector of embodiment 30 wherein the intratumoral electroporation pulse comprises at least one low voltage intratumoral electroporation (IT-EP) treatment.

32. The expression vector of embodiment 31, wherein the IT-EP treatment comprises at a field strength from 200 V/cm to 500 V/cm and a pulse length of about 100 μs to about 50 ms.

33. The expression vector of embodiment 32 wherein the treatment is one IT-EP treatment and comprises a field strength of at 350-450 V/cm and a pulse length of about 10 ms.

34. The expression vector of embodiment 33 wherein the treatment is one IT-EP treatment and comprises a field strength of about 400 V/cm and a pulse length of about 10 ms.

35. The expression vector of any of embodiments 30-34 wherein the treatment comprises 1-10 10 ms electroporation pulses.

36. The expression vector of embodiment 35 wherein the treatment comprises 5-10 10 ms electroporation pulses.

37. The expression vector of embodiment 36 wherein the treatment comprises 8 10 ms electroporation pulses.

38. An expression plasmid comprising a plurality of expression cassettes defined by the formula:

P-A-T-A′-T-B

wherein:

a) P is a human CMV promoter;

b) A and A′ are interleukin-12 (IL-12) p35 and p40, respectively;

c) B is FLT-3L fused to at least one antigen from Table 1; and

d) T is a P2A translation modification element.

39. The expression vector of embodiment 38 wherein the antigen is selected from the group consisting of: NYESO-1, OVA, RNEU, MAGE-A1, MAGE-A2, Mage-A10, SSX-2, Melan-A, MART-1, Tyr, Gp100, LAGE-1, Survivin, PRS pan-DR, CEA peptide CAP-1, OVA, HCV-NS3, TERT, WT1, PSMA, and an HPV vaccine peptide

40. The expression vector of embodiment 39, wherein the antigen is NYESO-1.

41. The expression vector of embodiment 38, comprising a nucleic acid sequence of SEQ ID NO: 1.

42. A method of treating a tumor in a subject comprising delivering the expression plasmid of any of embodiments 38-41 into the tumor using at least one intratumoral electroporation pulse.

43. The method of embodiment 42, wherein the intratumoral electroporation pulse has a field strength of about 200 V/cm to 1500 V/cm.

44. The method of embodiment 42 or 43, wherein the subject is a human.

45. The method of any of embodiments 42-44, wherein the tumor is selected from the group of melanoma, triple negative breast cancer, Merkel Cell Carcinoma, CTCL, and head and neck squamous cell carcinoma.

46. The method of any of embodiments 42-45, wherein the electroporation pulse is delivered by a generator capable of electrochemical impedance spectroscopy.

47. A method of treating a tumor in a subject comprising at least one low voltage intratumoral electroporation (IT-EP) treatment delivering an expression plasmid encoding interleukin-12 (IL-12), wherein the plasmid contains a P2A exon skipping motif.

48. The method of embodiment 47, wherein the IT-EP treatment comprises at a field strength from 200 V/cm to 500 V/cm and a pulse length of about 100 μs to about 50 ms.

49. The method of embodiment 48 wherein the treatment is one IT-EP treatment and comprises a field strength of at least 400 V/cm and a pulse length of about 10 ms.

50. The method of any of embodiments 47-49, wherein the low voltage IT-EP treatment of the IL-12 encoded plasmid containing P2A comprises at least one of the following when compared to an IL-12 encoded plasmid containing an IRES motif:

a) at least 3.6 times higher intratumoral expression of IL-12;

b) a lower mean tumor volume in a treated tumor lesion;

c) a lower mean tumor volume in an untreated contralateral tumor lesion;

d) a higher influx of lymphocytes into the tumor;

e) an increase of circulating tumor-specific CD8+ T cells;

f) an increase of lymphocyte and monocyte cell surface marker expression in the tumor; and

g) an increase in mRNA levels of INF-g related genes of Tables 23 and 24.

### Sequence Identifiers

TABLE 31
Sequence Identifier Table

| SEQ ID NO | Description |
|---|---|
| 1 | pOMIP2A-IL12-FLT3L-NYESO1 (OMI-PIIM)(DNA) |
| 2 | Human IL-12p35-P2A (protein) |
| 3 | Human IL-12p40-P2A (protein) |
| 4 | FLT3L- NYESO1 (amino acids 80-180) fusion protein |
| | (protein) |
| 5 | Human IL12p35-[GSG Hinge]-P2A (nucleotide) |
| 6 | Human IL12p40-[GSG Hinge]-P2A (nucleotide) |
| 7 | [IgK signal peptide]-Flt3L-[GSSGSSG Hinge]- |
| | NY-ESO1(80-180aa) (nucleotide) |
| 8 | hIL12p35-[GSG-Hinge]-P2A-hIL12p40 (nucleotide) |
| 9 | Human IL-12p35 - P2A - Human IL-12p40 (protein) |
| 10 | hIL12p35-[GSG Hinge]-P2A-p40-[GSG-Hinge]- P2A- |
| | [IgK signal peptide]-Flt3L-[GSSGSSG Hinge]-NY- |
| | ESO1(80-180aa) (nucleotide) |
| 11 | hIL12p35-[GSG Hinge]-P2A-p40-[GSG-Hinge]- P2A- |
| | [IgK signal peptide]-Flt3L-[GSSGSSG Hinge]-NY- |
| | ES01(80-180aa) (protein) |
| 12 | CMV-hIL12p35-P2A-hIL12p40-Flt3L-NYESO- |
| | 1(80-180aa) (nucleotide) |

The above provided embodiments and items are now illustrated with the following, non-limiting examples.

### Examples

### I. General Methods.

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

II. Subcloning of Human IL-12 p35 and p40 Subunits into pOMIP2A

A pUMVC3 backbone was purchased from Aldevron (Fargo, N. Dak.). A 1071 bp DNA fragment (gene block) encoding the translation modulating element P2A linked in-frame to hIL12p40 (P2A-hIL12p40) was purchased from IDT (Coralville, Iowa). The p40 geneblock was PCR amplified using Phusion polymerase (NEB, Ipswich Mass., cat. # M0530S) and ligated into pUMVC3 downstream of the CMV promoter/enhancer using standard restriction enzyme pairing and T4 DNA ligase (Life Technologies, Grand Island N.Y., cat. #15224-017). Positives clones of P2A-hIL12p40/pOMIP2A were identified via restriction enzyme digests and verified with DNA sequencing.

Human p35 was ordered as a 789 bp geneblock from IDT (Coralville Iowa) with internal BamH1, BglII and Xba1 sites removed to facilitate cloning. The p35 geneblock was PCR amplified as described above and ligated upstream of the p40 geneblock in P2A-hIL12p40/pOMIP2A. Positives clones of hIL12p35-P2A-p40/pOMIP2A were identified via restriction enzyme digests and verified with DNA sequencing.

A similar construct was made containing reporter genes for in vivo imaging and ex-vivo flow cytometry. For generation of pOMI-Luc2p-P2A-mCherry, Luc2P was PCR amplified from pGL4.32[luc2P/NF-κB-RE/Hygro] (Promega) and mCherry was amplified from a gene block fragment (IDT). Amplified DNA fragments were purified, digested and ligated into pUMVC3. Positive clones were identified via restriction enzyme digests and verified with DNA sequencing.

### III. Generation of FLT3L-Antigen Fusion Protein Constructs

The FMS-like tyrosine kinase 3 ligand (FLT3L) has been shown to direct antigen to antigen-presenting cells (APC) for preferential presentation to T cells (Kim et al. Nat Comm. 2014, Kreiter et al., Cancer Res. 2011, 71:6132). A soluble, secreted form of FLT3L is fused to a variety of protein or peptide antigens (Table 1; Kim et al. Nat Comm. 2014).

An example protocol is given for generating a FLT3L-NY-ESO-1 fusion protein construct. Three gene blocks were obtained from IDT that each contained the IgK signal peptide sequence followed by the ECD of FLT3L, a short hinge region, and three different segments of the NY-ESO-1 antigen. PCR was used to add flanking restriction sites and introduce these three fusion protein constructs into pUMVC3. FLT3L was also fused to a concatamer of 3 peptides containing the SIINFEKL peptide antigen from the ovalbumin gene for pre-clinical studies in mice. From pUMVC3, these fusion constructs are introduced into pOMIP2A (described below).

An alternative fusion protein using other shared tumor or viral antigens (Table 1) is constructed using the same method.

In addition to identified shared tumor antigens, patient-specific neoantigens could be identified and immunogenic peptide antigens tailored to that patient can be fused to FLT3L for personalized therapy via intratumoral electroporation, (see, e.g., Beckhove et al., J. Clin. Invest. 2010, 120:2230).

Versions of all immune-modulatory proteins are constructed in parallel using mouse homolog sequences and are used in pre-clinical studies.

IV. Generation of pOMI-2xP2A for Expression of Three Proteins from a Single Transcript.

An example subcloning protocol is given for IL-12 heterodimeric cytokine, and FLT3L-NY-ESO-1. A DNA geneblock (IDT) encoding FLT3L-NYESO-1 was PCR-amplified with an upstream P2A site and flanking restriction sites and ligated downstream of hIL-12p40. Quikchange mutagenesis (Agilent, Santa Clara, USA) was performed to delete the stop site 3′ of p40. Positives clones were identified via restriction enzyme digests and verified with DNA sequencing.

A forth gene can be added either upstream or downstream of the three genes already in the polycistronic message using the same methods.

V. Generation of pOMI-PIIM

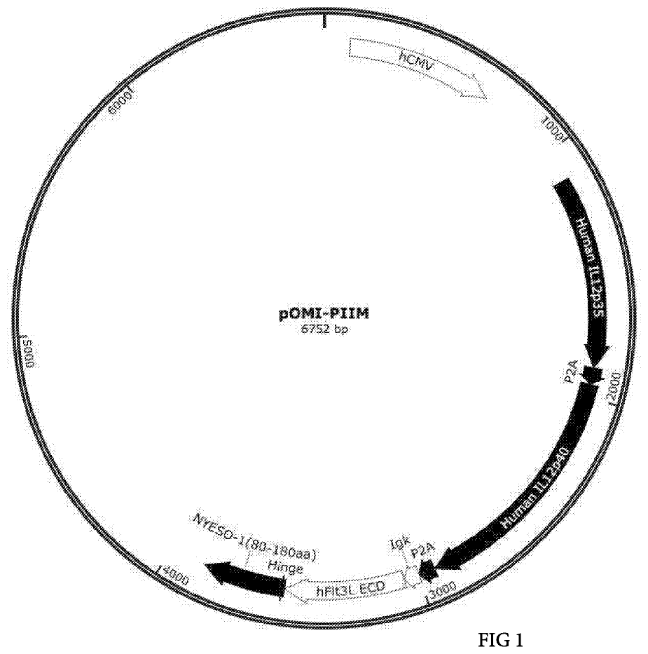

A schematic diagram of the pOMI-PIIM plasmid is shown in FIG. 1. OMI-PIIM stands for OncoSec Medical Incorporated-Polycistronic IL-12 Immune Modulator. All three genes are expressed from the same promoter, with intervening exon skipping motifs to allow all three proteins to be produced from a single polycistronic message.

The vector pUMVC3 was linearized by Kpn1 restriction enzyme digest. hIL12p35 was amplified by PCR from the clinical hIL12-IRES/pUMVC3 plasmid Aldevron (Fargo, N. Dak.) with 24 bp overlap matching the 5′ sequence of linearized pUMVC3 and a 3′ partial P2A sequence. hIL12p40 was amplified by PCR from the hIL12-2A/pUMVC3 plasmid (described above) with a 5′ P2A sequence and 3′ 24 bp overlap with linearized pUMVC3. The sequence overlap between the p35-P2A (partial) and P2A-p40 PCR products was 14 bp. Gibson assembly of the three pieces was performed per the manufacturer's recommendations (New England Biolabs E2611S/L) and positive clones of hIL12-2A-seamless/pUMVC3 were screened by restriction enzyme digests and verified by DNA sequencing.

Subsequently, this construct was digested with Not1 to linearize it 3′ of the hIL12p40 stop site. Using hIL12˜hFLT3L-NYESO1 as a template (described above), P2A-FLT3L-NYESO (80-180aa) was amplified by PCR with a 5′ 28 bp overlap with the end of hIL12p40 (deleting the stop site) and a 3′ 28 bp overlap with linearized pUMVC3. Gibson assembly (New England Biolabs E2611S/L) was performed per the manufacturer's recommendations and positive clones of hIL12˜hFLT3L-NYESO (80-180aa)-seamless/pUMVC3 were screened by restriction enzyme digests and verified by DNA sequencing (pOMI-PIIM, Sequence ID #1).

A mutant form of FLT3L that fails to bind the FLT3 receptor was generated as a control for functional studies (Graddis et al., 1998, J. Biol. Chem. 273:17626). Quikchange mutagenesis (Agilent, Santa Clara, USA) was used to create point mutations as described in Graddis (supra) and pOMI-PIIM as a template.

In parallel, a version of pOMI-PIIM was constructed with mouse IL-12 for pre-clinical studies.

### VI. ELISA

pUMVC3-IL12 (Aldevron, Fargo, N. Dak.) and pOMI-IL12P2A were transfected into HEK293 cells using TransIT LT-1 (Mirus, Madison Wis., cat. # MIR 2300) according to the manufacturers recommendations. Two days later, supernatants were collected and spun for 5 minutes at 3000 rpm to remove any cell debris. Cleared supernatants were aliquoted and frozen at −86° C. The levels of hIL-12p70 heterodimeric proteins in the conditioned media were quantitated using an ELISA that specifically detects the complexes (R&D Systems, Minneapolis Minn. cat. # DY1270).

TABLE 4
Relative expression of hIL-12p70 protein from culture supernatants
of cells transfected with pOMI-IL12P2A and pUMVC3-IL12

| Plasmid | hIL-12p70 (ng/ml) Mean +/− SEM n = 2 |
|---|---|
| No transfection control | 2.0 +/− 2.0 |
| pUMVC3-hIL12 | 442.4 +/− 181.6 |
| pOMI-IL12P2A | 1603.4 +/− 77.4 |

pOMI-IL12P2A generated 3.6 times more human IL12p70 secreted protein than did pUMVC3-IL12 in culture supernatants for a given amount of transfected plasmid.

Clones of pOMI-PIIM were transfected into HEK293 cells using TransIT LT-1 (Mirus, Madison Wis., cat. # MIR 2300) according to the manufacturers recommendations. Two days later, supernatants were collected and spun for 5 minutes at 3000 rpm to remove any cell debris. Cleared supernatants were transferred to new tubes, aliquoted and frozen at −86° C. The levels of hIL-12p70 heterodimeric proteins in the conditioned media were quantitated using an ELISA that specifically detects the complexes (R&D Systems, Minneapolis Minn. cat. # DY1270). The level of FLT3L-NYESO-1 fusion protein was quantified by ELISA with anti-FLT3L antibodies (R&D Systems, Minneapolis Minn. cat. # DY308).

A significant level of both p70 IL-12 and FLT3L fusion proteins were produced from cells transfected with pOMI-PIIM (Table 5)

TABLE 5
Expression and secretion of IL-12 p70 and FLT3L-
NYESO1 fusion protein from cells transfected with
pOMI-PIIM were measured by ELISA and are shown.

| Secreted protein | ng/ml; Mean +/− SEM |
|---|---|
| IL-12 p70 | 1364 +/− 5.5 |
| FLT3L-NY-ESO-1 fusion protein | 25.1 +/− 3.1 |

### VII. In Vitro Functional Assays

Tissue culture supernatants from cells expressing pOMI-IL12P2A and pOMI-PIIM were tested for the expression of functional IL-12 p70 using HEK-Blue cells. These cells are engineered to express human IL-12 receptors, and a STAT4-driven secreted form of alkaline phosphatase.

This reporter assay was performed according to the manufacturer protocol (HEK-Blue IL-12 cells, InvivoGen catalog # hkb-i112). Expression of secreted alkaline phosphatase (SEAP) was measured according to the manufacturer's protocol (Quanti-Blue, InvivoGen catalog # rep-qbl).

Different dilutions of culture supernatants from HEK293 cells transfected with the same amount of either human pOMI-IL12P2A or pUMVC3-IL12 (Aldevron) were compared in this assay. The mean EC50 was >2-fold lower in pOMI-IL12P2A samples (n=3, Mann-Whitney; ** p<0.01) These data show that for a given dose of plasmid, pOMI-IL12P2A resulted in production of more functional human IL-12p70 protein than did pUMVC3-IL12.

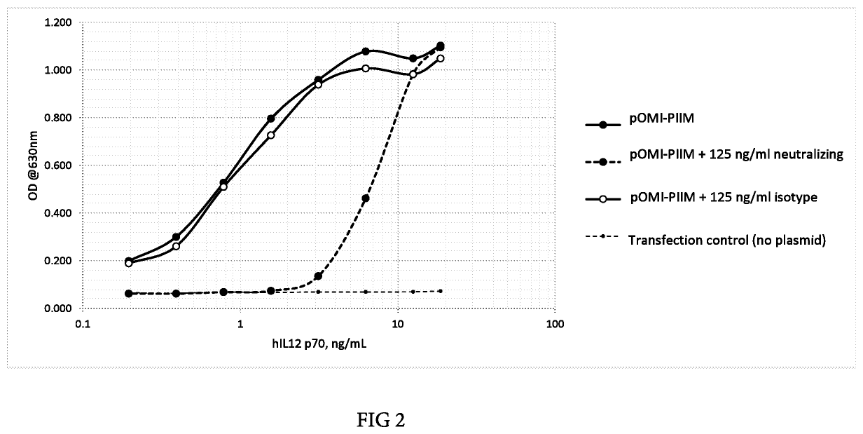

IL-12 p70 protein expressed and secreted from the pOMI-PIIM polycistronic vector also demonstrated strong activity in the induction of SEAP protein (FIG. 2). This activity was comparable to rhIL-12 protein controls, and was blocked by a neutralizing IL-12 antibody (R&D systems; AB-219-NA) (FIG. 2).

Human FLT3L and FLT3L-NYESO1 fusion proteins expressed from pOMIP2A vectors and secreted into the culture medium of HEK 293 cells were tested for binding to FLT3 receptors expressed on the surface THP-1 monocytic cells.

HEK cells were transfected with pOMIP2A-hFLT3L or pOMIP2A-hFLT3L-NYESO1 (80-180aa) using Minis TransIT LT-1. Supernatants were collected after 72 hours. The amount of secreted FLT3L proteins was quantified using hFLT3L ELISA (R&D Systems cat. # DY308).

The THP-1 monocyte cell line was cultured in RPMI+10% FBS+1% P/S (ATCC, cat. # TIB-202). For each experiment, 750,000 THP-1 cells were washed in Fc buffer (PBS+5% filtered FBS+0.1% NaN3), preincubated with human Fc block (TruStain FcX, Biolegend 422301) for 10 minutes and then incubated with 150 ng of recombinant hFLT3L-Fc (R&D Systems, cat. # AAA17999.1) or HEK 293 conditioned media containing 150 ng hFLT3L or hFLT3L-NYESO1 protein and incubated for 1 hour at 4° C. Cells were then washed in Fc buffer and incubated with biotinylated anti-hFLT3L antibodies (R&D Systems, cat. # BAF308) for 1 hour. Cells were then washed in Fc buffer and incubated with streptactin-AlexaFluor-647 2° Ab for 1 hr (ThermoFisher, # S32357). Cell were washed again and analyzed by flow cytometry using a Guava 12HT cytometer (Millipore) on the Red-R channel. HEK 293 cells which do not express FLT3 receptors were also tested as a negative control.

TABLE 6
Secreted recombinant FLT3 ligand proteins bind to
FLT3 receptors of the surface of THP-1 monocytes

| | Mean fluorescence intensity | | | |
|---|---|---|---|---|
| Cell line | unstained | Control super | hFLT3L | hFLT3L-NYESO1 |
| THP-1 | 9.0 | 9.7 | 32.2 | 52.2 |
| HEK293 | 9.0 | 7.5 | 8.4 | 8.8 |

Over 90% of THP-1 cells showed an increase in mean fluorescence intensity with both hFLT3L and hFLT3L-NYESO1 fusion proteins expressed from pOMIP2A vectors indicating that these recombinant proteins bind efficiently to FLT3 receptors on the cell surface (Table 6).

In order to further test the functionality of the recombinant FLT3L proteins, HEK 293 conditioned media were used to test for induction of dendritic cell maturation in mouse splenocytes.

Spleens were excised from B16-F10 tumor bearing C58/BL6 mice. Under sterile conditions, spleens were placed in DMEM media into the 70-micron cell strainer (Miltenyi) and mechanically dissociated using the rubber tip of the plunger from a 3 ml syringe. Once the spleen is completely dissociated, 10 mls of HBSS with 10% FBS (PFB) wad used to wash the strainer. Flow-though was spun in a centrifuge at 300×g for 10 mins. to pellet cells. Cells were washed once with PFB. Red blood cells were lysed with ACK lysis buffer according to the manufacturer's instructions (Thermo Fisher A1049201). Cells were filtered through a 40-micron cell strainer into a 15 ml conical tube and spun in a centrifuge at 300×g. Single cell suspension from the spleens were resuspended in complete RPMI-10 media. 1.5 million splenocytes were plated in a 12 well plate and allowed to adhere to the plate approximately 3 hrs. Non-adherent cells were removed and 2 mls of complete RPMI-10 media containing murine GMCSF (100 ng/ml) and murine IL-4 (50 ng/ml) were added. The media was changed every 2 days for a week. The adherent dendritic cells were treated in triplicate wells with 1 ml of HEK 293 conditioned supernatants (containing 100 ng/ml Flt3L-NYESO1 fusion protein) for 7 days.100 ng of human FLT3 ligand recombinant protein was compared as a positive control (R&D systems, AAA17999.1). Cells were gently scraped from a plate and the number of CD11c⁺ cells was determined by flow cytometric analysis.

When the number of CD3 (−) CD11c (+) dendritic cells was tabulated, conditioned media from cells transfected with pOMI-FLT3L-NYESO1 plasmid generated a significant increase in the number of these cells as compared to splenocytes incubated with conditioned media from un-transfected cells.

This result indicated that the FLT3L-NYESO1 fusion protein could function to stimulate FLT3 receptor-mediated dendritic cell maturation ex-vivo in mouse splenocytes.

### VIII. Tumors and Mice

Female C57Bl/6J or Balb/c mice, 6-8 weeks of age were obtained from Jackson Laboratories and housed in accordance with AALAM guidelines.

B16-F10 cells were cultured with McCoy's 5A medium (2 mM L-Glutamine) supplemented with 10% FBS and 50 μg/ml gentamicin. Cells were harvested with 0.25% trypsin and resuspended in Hank's balanced salt solution (HBSS). Anesthetized mice were subcutaneously injected with 1 million cells in a total volume of 0.1 ml into the right flank of each mouse. 0.25 million cells in a total volume of 0.1 ml were injected subcutaneously into the left flank of each mouse.

Tumor growth was monitored by digital caliper measurements starting day 8 until average tumor volume reaches ˜100 mm³. Once tumors are staged to the desired volume, mice with very large or small tumors were culled. Remaining mice were divided into groups of 10 mice each, randomized by tumor volume implanted on right flank.

Additional tumor cell types were tested including B16OVA in C57Bl/6J mice as well as CT26 and 4T1 in Balb/c mice.

This protocol was used as a standard model to test simultaneously for the effect on the treated tumor (primary) and untreated (contralateral). Lung metastases were also quantified in Balb/c mice bearing 4T1 tumors.

### IX. Intratumoral Treatment

Mice were anesthetized with isoflurane for treatment. Circular plasmid DNA was diluted to 1 μg/μ1 in sterile 0.9% saline. 50 μl of plasmid DNA was injected centrally into primary tumors using a 1 ml syringe with a 26 Ga needle. Electroporation was performed immediately after injection. Electroporation of DNA was achieved using a Medpulser with clinical electroporation parameters of 1500 V/cm, 100 μs pulses, 0.5 cm, 6-needle electrode. Alternative parameters used were 400 V/cm, 10-ms pulses, using either a BTX generator or a generator incorporating impedance spectroscopy, as described above. Tumor volumes were measured twice weekly. Mice were euthanized when the total tumor burden of the primary and contralateral reached 2000 mm³.

X. Intratumoral expression

In vivo Imaging. An optical imaging system (Lago, Spectral Instruments) was used to quantify luminescence of tumors that were previously treated with pOMI-Luc2p-P2A-mCherry plasmid. The mice were imaged at different time points. To perform imaging, animals were anesthetized by exposed to 2% isoflurane in 500 ml/min of oxygen. Once anesthetized, 200 μl of a 15 mg/ml solution of D-luciferin (Gold Bio) prepared in sterile D-PBS was administered by intraperitoneal injection with a 27-gauge syringe. Animals were then transferred to an anesthesia manifold on a 37° C. heated stage, where they continued to receive 2% isoflurane in 500 ml/min of oxygen. Luminescent images were acquired 20 minutes after injection using a 5 s exposure to a CCD camera cooled to −90° C. Total photons emitted from each tumor was determined by post-processing using a region of interest with a 0.5 cm radius (AmiView, Spectral Instruments).

TABLE 7
Relative expression of Luciferase in tumors 48
hours after electroporation with 1500 V/cm,
6 0.1 ms pulses vs. 400 V/cm, 8 10 ms pulses

| | Photons/second; |
|---|---|
| Intratumoral treatment | mean ± SEM n = 11 |
| OMI-Luc2p-P2A-mCherry/no EP | 37,389 ± 8146 |
| OMI-Luc2p-P2A-mCherry/EP | 794,900 ± 182,843 |
| 1500 V/cm 0.1 ms | |
| OMI-Luc2p-P2A-mCherry/EP | 7,937,411 ± 2,708,234 |
| 400 V/cm 10 ms | |

Introduction of the pOMI-Luc2p-P2A-mCherry plasmid with EP under low voltage conditions lead to nearly a 10-fold higher level of luciferase activity in electroporated tumors as visualized with in vivo imaging (Table 7).

Dissociation of Tumors for Flow Cytometric Analysis.

Single cell suspensions were prepared from B16-F10 tumors. Mice were sacrificed with CO₂ and tumors were carefully excised leaving skin and non-tumor tissue behind. The excised tumors were then stored in ice-cold HBSS (Gibco) for further processing. Tumors were minced and incubated with gentle agitation at 37° C. for 20-30 min in 5 ml of HBSS containing 1.25 mg/ml Collagenase IV, 0.125 mg/ml Hyaluronidase and 25 U/ml DNase IV. After enzymatic dissociation, the suspension was passed through a 40 μm nylon cell strainer (Corning) and red blood cells removed with ACK lysis buffer (Quality Biological). Single cells were washed with PBS Flow Buffer (PFB: PBS without Ca⁺⁺ and Mg⁺⁺ containing 2% FCS and 1 mM EDTA) pelleted by centrifugation and resuspended in PFB for immediate flow cytometric analysis.

TABLE 8
Relative percentage of isolated tumor cells and tumor infiltrating
lymphocytes (TIL) that express RFP (mCherry) protein 48 hours
after IT-EP as visualized with flow cytometry

| Intratumoral Treatment | % RFP⁺ cells of all live cells |
|---|---|
| Untreated control | 0.00 +/− 0.00 |
| OMI-Luc2p-P2A-mCherry/no EP | 0.24 +/− 0.03 |
| OMI-Luc2p-P2A-mCherry/EP | 2.04 +/− 0.53 |
| 1500 V/cm 0.1 ms | |
| OMI-Luc2p-P2A-mCherry/EP | 8.16 +/− 0.92 |
| 400 V/cm 10 ms | |

As seen using the RFP reporter gene, high voltage conditions resulted in ˜2% of the tumor cells expressing the protein, and low voltage, longer pulse condition resulted in >8% of the cells expressing the protein. The percentage with low voltage conditions is approaching the transduction efficiency of viral vectors (Currier, M. A. et al., Cancer Gene Ther 12, 407-416, doi:10.1038/sj.cgt.7700799 (2005).

Tumor Lysis for Protein Extraction.

One, 2 or 7 days after IT-EP (400 v/cm, 8 10-ms pulses), tumor tissue was isolated from sacrificed mice to determine expression of the transgenes. Tumor were dissected from mice and transferred to a cryotube in liquid nitrogen. The frozen tumor was transferred to a 4 ml tube containing 300 μL of tumor lysis buffer (50 mM TRIS pH 7.5, 150 mM NaCl, 1 mM EDTA, 0.5% Triton X-100, Protease inhibitor cocktail) and placed on ice and homogenized for 30 seconds (LabGen 710 homogenizer). Lysates were transferred to 1.5 ml centrifuge tube and spun at 10,000×g for 10 minutes at 4° C. Supernatants were transferred to a new tube. Spin and transfer procedure was repeated three times. Tumor extracts were analyzed immediately according to manufacturer's instruction (Mouse Cytokine/Chemokine Magnetic Bead Panel MCYTOMAG-70K, Millepore) or frozen at −80° C. Recombinant Flt3L-OVA proteins were detected by standard ELISA protocols (R&D systems) using anti-FLT3L antibody for capture (R&D Systems, Minneapolis Minn. cat. # DY308) and an Ovalbumin antibody for detection (ThermoFisher, cat. # PA1-196).

TABLE 9
Intratumoral expression of hIL-12 cytokine after electroporation of a pOMI
polycistronic plasmid encoding hIL-12 under low voltage conditions.

| | Untreated | | | EP/pOMI-hIL12/hIL15/hINF-γ | | |
|---|---|---|---|---|---|---|
| Recombinant | [Protein] pg/mg | | | [Protein] pg/mg | | |
| protein | Mean +/− SEM n = 2 | | | Mean +/− SEM n = 3 | | |
| detected | Day 1 | Day 2 | Day 7 | Day 1 | Day 2 | Day 7 |
| IL-12 p70 | 0 | 0 | 0 | 3000.5 ± 1872.7 | 2874.7 ± 1459.1 | 19.1 ± 4.2 |

To test for expression and function of our FLT3L-tracking antigen-fusion protein, we constructed a fusion of FLT3L (extracellular domain) and peptides from the ovalbumin gene in OMIP2A vectors and electroporated intratumorally as above.

TABLE 10
Intratumoral expression of FLT3L-OVA fusion protein (genetic
adjuvant with shared tumor antigen) 2 days after electroporation
under low voltage conditions as analyzed by ELISA (n = 8).

| | EP/pUMVC3 control | EP/pOMI-FLT3L-OVA |
|---|---|---|
| Recombinant protein | Mean +/− SEM | Mean +/− SEM |
| construct | pg/ml | pg/ml |
| FLT3L-OVA fusion | 30.6 +/− 1.4 | 441 +/− 102 |

After intratumoral electroporation of pOMIP2A vectors containing mouse homologs of the immunomodulatory proteins, significant levels of IL-12p70 (Table 9) and FLT3L-OVA recombinant proteins (Table 10) were detectable in tumor homogenates by ELISA.

### XI. Tumor Regression

OMIP2A plasmids were generated in parallel that contain mouse 11-12 and were used to test for in vivo biological activity in terms of tumor regression and changes to the host immune system in pre-clinical mouse models.

The protocol described above for creating mice with two tumors on opposite flanks was used as a standard model to test simultaneously for the effect on the treated tumor (primary) and untreated (contralateral). Lung metastases were also quantified in Balb/c mice bearing 4T1 tumors.

TABLE 11
Comparison of B16-F10 tumor regression for primary (treated)
and contralateral (untreated, distant) tumors after injection
of 50 μg of pOMI-IL12P2A vs. pUMVC3-IL12 (Aldevron) vs.
pUMVC3 control plasmids and IT-EP at 1500 volts/cm, 6, 0.1
ms pulses on Day 8, 12, and 15 after tumor cell inoculation.

Tumor volume (mm³) on Day 16
Mean +/− SEM, n = 10

| Intratumoral treatment | Primary tumor | Distant tumor |
|---|---|---|
| Untreated | 1005.2 +/− 107.4 | 626.6 +/− 71.8 |
| pUMVC3 control 50 μg | 345.2 +/− 130.5 | 951.1 +/− 77.0 |
| pUMVC3-mIL12 50 μg | 140.3 +/− 49.8 | 441.0 +/− 80.8 |
| pOMI-mIL12P2A 50 μg | 92.1 +/− 38.7 | 283.3 +/− 87.2 |

Data in Table 11 illustrate that IT-EP using the new plasmid design expressing IL12 subunits with the P2A exon skipping motif compared to the use of the internal ribosomal entry site (IRES), at high voltage, gave better control of tumor growth (both treated primary and distant untreated tumors) as expected with more efficient expression (Table 4).

TABLE 12
Comparison of B16-F10 tumor regression for primary and distant tumors
after IT-EP at 1500 volts/cm, 6 0.1-ms pulses vs. 400 V/cm, 8 10-ms
pulses on Day 8, 12, and 15 after tumor cell inoculation.

| | Tumor volume (mm³) on Day 16 | |
|---|---|---|
| | Mean +/− SEM, n = 10 | |
| Intratumoral treatment | Primary tumor | Distant tumor |
| Untreated | 1005.2 +/− 107.4 | 626.6 +/− 71.8 |
| pUMVC3/EP 1500 V/cm 0.1 ms | 345.2 +/− 130.5 | 951.1 +/− 77.0 |
| pUMVC3-mIL12 1500 | 140.3 +/− 49.8 | 441.0 +/− 80.8 |
| V/cm 0.1 ms | | |
| pUMVC3/EP 400 V/cm 10 ms | 437.3 +/− 130.2 | 943.7 +/− 143.7 |
| pUMVC3-mIL12 400 | 131.5 +/− 31.6 | 194.5 +/− 39.6 |
| V/cm l0 ms | | |

Data in Table 12 show that when electroporation was performed with lower voltage, longer pulse conditions, better tumor growth inhibition in both an electroporated tumor lesion as well as a distant untreated lesion was seen, particularly in the distant, untreated tumor. These data suggested superior systemic tumor immunity was generated as compared to higher voltage, shorter pulse conditions.

Using the new plasmid design and lower voltage EP parameters, we tested different doses of pOMI-IL12P2A plasmid after just one dose on Day 10 after tumor cell inoculation.

TABLE 13
B16-F10 tumor regression for primary and distant tumors after
IT-EP with different doses of OMI-mIL12P2A. Electroporation
with the parameters of 400 V/cm, 8 10-ms pulses using acupuncture
needles was performed once, 10 days after implantation.

| | |
|---|---|
| | Tumor volume (mm³) on Day 19, |
| Plasmid dose | Mean +/− SEM, n = 10 |

| introduced by IT-EP | Primary tumor | Distant tumor |
|---|---|---|
| pUMVC3 control 50 μg | 556.4 +/− 59.0 | 211.3 +/− 46.5 |
| pOMI-mIL12P2A 1 μg | 546.1 +/− 92.5 | 158.4 +/− 47.1 |
| pOMI-mIL12P2A 10 μg | 398.6 +/− 78.4 | 79.7 +/− 18.7 |
| pOMI-mIL12P2A 50 μg | 373.6 +/− 46.3 | 74.3 +/− 12.1 |

The extent of regression of both primary, treated and distant, untreated tumors increased with electroporation of increasing dose of pOMI-mIL12P2A plasmid. With pOMI-IL12P2A, 10 μg of plasmid was sufficient for maximal effect and there was significant tumor growth control with a single dose of treatment with the new plasmid design and lower voltage electroporation conditions.

TABLE 14
Direct comparison of 10 μg pOMI-mIL12P2A/Low Voltage
EP with 10 μg pUMVC3-IL12/High Voltage EP in a
contralateral tumor regression model. Tumors were
treated once on Day 10 post tumor cell inoculation.

| | |
|---|---|
| | Tumor volume (mm³) on Day 18 |
| Intratumoral | Mean +/− SEM, n = 6 |

| treatment | Primary tumor | Distant tumor |
|---|---|---|
| pUMVC3-mIL12 1500 | 604.8 +/− 178.6 | 309.0 +/− 36.9 |
| V/cm 0.1 ms | | |
| pOMI2A-mIL12 400 | 54.3 +/− 20.8 | 87.0 +/− 46.4 |
| V/cm 10 ms | | |

Both the primary (treated) and the contralateral (untreated) tumor in pIL12-P2A+Low Voltage treated mice showed enhanced suppression of tumor growth. The improved therapeutic effect of intratumoral electroporation pOMI-IL12P2A with EP an low voltage was also reflected in a statistically significant survival advantage (5/6 mice survived until end of study with pOMI-IL12P2A/lowV vs. 1/6 for pUMVC3-IL12/highV)

The data in Table 14 show that with the new plasmid design coupled with the optimized electroporation parameters, significant tumor growth control, as well as systemic tumor immunity as measured by effects on contralateral, untreated tumors was achieved with a single EP treatment.

The ability of IT-EP of pOMI-mIL12P2A to affect 4T1 primary tumor growth and lung metastases in Balb/c mice was also tested.

One million 4T1 cells were injected subcutaneously on the right flank of the mice and 0.25 million 4T1 cells were injected into the left flank. Larger tumors on the right flank were subject to IT-EP with empty vector (pUMVC3, Aldevron) or with pOMI-mIL12P2A. Tumor volumes were measured every two days and on Day 19, mice were sacrificed, and the lungs were excised and weighed.

TABLE 15
Primary tumor growth and post-mortem weight of lungs of mice
electroporated with 400 V/cm, 8 10-ms pulses with acupuncture
needles on day 8, and day 15 post-implantation. Primary tumor
volumes were measured on Day 17, and lung weights on Day 18.

| | Primary tumor | Lung weight |
|---|---|---|
| | volume (mm³) | (grams) |
| | Mean +/− SEM, | Mean +/− SEM, |
| Treatment | n = 5 | n = 5 |
| Untreated | 897 +/− 131 | 0.252 +/− 0.019 |
| EP/pUMVC3 | 593 +/− 27 | 0.228 +/− 0.006 |
| EP/pOMIP2A- | 356 +/− 80 | 0.184 +/− 0.004 |
| mIL12 | | |

It has been previously reported that systemic IL-12 treatment can reduce lung metastases in mice with 4T1 tumors (Shi et al., J Immunol. 2004, 172:4111). Our finding indicates that local IT-EP treatment of the tumors also reduced metastasis of these tumor cells to the lung in this model (Table 15).

In addition to B16F10 tumors, electroporation of pOMI-mIL12P2A also resulting in regression of both primary (treated) and contralateral (untreated) B16OVA and CT26 tumors. In the 4T1 tumor model, the primary tumor regressed after EP/pOMI-mIL12P2A, and the mice demonstrated a significant reduction in lung weight, indicating a reduction in lung metastases. We show that IT-EP of OMI-mIL12P2A can reduce tumor burden in 4 different tumor models in two different strains of mice.

TABLE 16
B16-F10 tumor regression for treated and untreated tumors
after intratumoral electroporation of pOMIP2A plasmids
containing genes encoding mIL-12 and FLT3L-OVA using 400
V/cm, and 8 10-ms pulses on day 7 and 14 after tumor cell
inoculation; tumors measurements shown from Day 16.

Tumor volume (mm³),
Mean +/− SEM, n = 10

| Treatment | Primary tumor | Distant tumor |
|---|---|---|
| EP/pUMVC3 control | 600.7 +/− 113.3 | 383.4 +/− 75.9 |
| EP/pOMI-IL12P2A + | 94.2 +/− 31.7 | 115.7 +/− 42.3 |
| pOMI-FLT3L-OVA | | |

TABLE 17
B16-F10 tumor regression for treated and untreated tumors
after IT-EP of pOMI-PIIM (version containing mouse IL-12)
using 400 V/cm, and 8 10-ms pulses on day 7 after tumor
cell inoculation; tumors measurements shown from Day 15.

Tumor volume (mm³), Mean +/− SEM

| Treatment | Primary tumor | Distant tumor |
|---|---|---|
| EP/pUMVC3 empty | 895.94 +/− 94.29 | 459.51 +/− 64.45 |
| vector n = 9 | | |
| EP/pOMI-PIIM n = 7 | 274.70 +/− 36.27 | 140.71 +/− 32.26 |

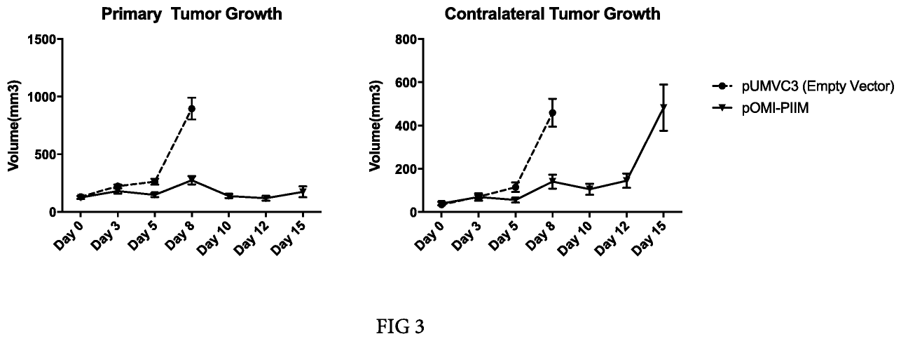

Electroporation of a pOMI-PIIM expressing both mouse IL-12 p70 and human FLT3L-NY-ESO-1 fusion protein caused significantly reduced growth of both the primary, treated and the distant, untreated tumors (Table 17 and FIG. 3) with only a single treatment.

The volume of both primary and contralateral tumors is significantly reduced in mice where immunomodulatory genes were introduced by electroporation as compared with electroporation of empty vector control, indicating not only a local effect within the treated tumor microenvironment, but an increase in systemic immunity as well.

### XII. Flow Cytometry

At various time points after IT-pIL12-EP treatment, mice were sacrificed and tumor and spleen tissue were surgically removed.

Splenocytes were isolated by pressing spleens through a 70-micron filter, followed by red blood cell lysis (RBC lysis buffer, VWR, 4203010BL), and lympholyte (Cedarlane CL5035) fractionation. Lymphocytes were stained with SIINFEKL-tetramers (MBL International T03002), followed by staining with antibody cocktails containing: anti-CD3 (Biolegend 100225), anti-CD4 (Biolegend 100451), anti-CD8a (Biolegend 100742), anti-CD19 (Biolegend 115546), and vital stain (live-dead Aqua; Thermo-Fisher L-34966). Cells were fixed and analyzed on an LSR II flow cytometer (Beckman).

Tumors were dissociated using Gentle-MACS for tumors (Miltenyi tumor dissociation kit 130-096-730, C-tubes, 130-093-237) and homogenized using a Miltenyi gentleMACS™ Octo Dissociator with Heaters (130-096-427). Cells were pelleted at 800×g for 5 min at 4° C. and re-suspended in 5 mL of PBS+2% FBS+1 mM EDTA (PFB) and overlaid onto 5 mL of Lympholyte-M (Cedarlane). Lympholyte columns were spun in centrifuge at 1500×g for 20 min at room temperature with no brake. Lymphocyte layer was washed with PBF. Cell pellets were gently re-suspended in 500 μL of PFB with Fc block (BD Biosciences 553142). In 96-well plate, cells were mixed with a solution of SIINFEKL tetramer (MBL), representing the immunodominant antigen in B16OVA tumors, according to the manufacturers instruction and incubated for 10 minutes at room temperature. Antibody staining cocktails containing the following: Anti-CD45-AF488 (Biolegend 100723), anti-CD3-BV785 (Biolegend 100232), Anti-CD4-PE (eBioscience12-0041), anti-CD8a-APC (eBioscience 17-0081), anti-CD44-APC-Cy7 (Biolegend 103028), anti-CD19-BV711 (Biolegend 11555), anti-CD127 (135010), anti-KLRG1 (138419), were added and incubated at room temperature for 30 minutes. Cells were washed 3 times with PFB. Cells were fixed in PFB with 1% paraformaldehyte for 1 minute on ice. Cells were washed twice with PFB and stored at 4° C. in the dark. Samples were analyzed on an LSR II flow cytometer (Beckman).

TABLE 18
Relative influx of lymphocytes in primary tumors after intratumoral
electroporation of OMI-mIL12P2A under low voltage conditions vs
pUMVC3-IL12 under high voltage conditions (n = 5 per cohort).

| Intratumoral | % CD45⁺ of | % CD8⁺ of all | CD4⁺/CD8⁺ |
|---|---|---|---|
| treatment | all live cells | live cells | ratio |
| pUMVC3-mIL12/ | 21.8 +/− 6.7 | 4.4 +/− 2.7 | 0.81 +/− 0.18 |
| EP 1500 V/cm | | | |
| 0.1 ms | | | |
| pOMIP2A-mIL12/ | 40.5 +/− 4.6 | 10.7 +/− 1.9 | 0.12 +/− 0.004 |
| EP 400 V/cm | | | |
| 10 ms | | | |

In addition to reducing tumor growth, pOMI-mIL12P2A/EP lowV also increased influx of lymphocytes in primary, treated tumors as compared to pUMVC3-mIL12/EP highV and decreased the CD4+/CD8+ ratio within the TIL population.

Systemic tumor immunity after pOMI-IL12P2A/EP low V treatment was further assessed in spleen and distant, untreated tumors.

TABLE 19
IT-pOMIP2A-mIL12-EP increased SIINFEKL-tetramer-binding CD8+
T cells in the spleens of treated, B16OVA tumor-bearing mice.
Mice were electroporated intratumorally (IT-EP) once on Day
10 after tumor cell inoculation using 400 V/cm, 10-ms pulses,
300 ms pulse frequency, with 0.5 cm acupuncture needles.

| | Percent of CD3⁺CD8⁺CD44⁺ T cells that are |
|---|---|
| Treatment | SIINFEKL-tetramer positive on Day 23, n = 6 |
| IT-pOMI-mIL12P2A-EP | 2.36 +/− 0.75 |
| IT-pUMVC3-EP | 0.24 +/− 0.04 |
| Untreated | 0.10 +/− 0.04 |

IT-pOMI-mIL12P2A-EP induces an increase in circulating CD8⁺ T cells directed against the SIINFEKL peptide from ovalbumin, the dominant antigen in B16OVA tumors. These data indicate that local IL-12 therapy can lead to systemic tumor immunity in mice.

TABLE 20
Intratumoral electroporation of OMI-mIL12P2A alters the immune
environment in B16OVA distant, untreated tumors. Mice were
electroporated intratumorally (IT-EP) once on Day 10 after
cell implantation using 400 V/cm, 10-ms pulses, 300 ms pulse
frequency, with 0.5 cm acupuncture needles. The composition
of infiltrating lymphocytes (TIL) in untreated tumors measured
18 days after treatment is shown.

| | Composition of TIL in distant, untreated | | |
|---|---|---|---|
| | tumors Mean +/− SEM, n = 6 | | |
| | % CD3⁺CD8⁺ | % SLEC | CD8⁺/T_{reg} T |
| Treatment | T cells | T cells | cell ratio |
| IT-pOMI- | 14.8 +/− 2.7 | 1.0 +/− 0.1 | 1892 +/− 602 |
| mIL12P2A-EP | | | |
| IT-pUMVC3-EP | 3.6 +/− 1.1 | 0.2 +/− 0.07 | 659 +/− 129 |
| Untreated | 2.9 +/− 0.9 | 0.09 +/− 0.03 | 753 +/− 288 |

Electroporation of OMI-mIL12P2A into the primary tumor can significantly alter the composition of TILs within the contralateral, untreated tumor (Table 20). These results show that intratumoral treatment with OMI-mIL12P2A can affect the immune environment in untreated tumors indicating that local treatment leads to a systemic anti-tumor immune response. This conclusion is corroborated by increased detection of tumor antigen-specific C8⁺ T cells in the spleen (Table 19), contralateral tumor regression (Tables 11,12,13,14), and reduction in lung metastases (Table 15).

### XIII. Analysis of Mouse Gene Expression

NanoString® was used for analysis of changes in gene expression in primary, treated and distant, untreated tumors induced by IT-EP of pOMI-mIL12P2A, pOMI-PIIM (version with mouse IL-12) and pOMI-FLT3L-NYESO1 plasmids. Tumor tissue was carefully harvested from mice using scalpel and flash frozen in liquid nitrogen. Tissues were weighed using a balance (Mettler Toledo, Model ML54). 1 ml of Trizol (Thermo Fisher Scientific, Waltham, Mass.) was added to the tissue and homogenized using a probe homogenizer on ice. RNA was extracted from Trizol using manufacturer's instructions. Contaminating DNA was removed by DNase (Thermo Fisher, Cat no: EN0525) treatment. Total RNA concentrations were determined using the NanoDrop ND-1000 spectrophotometer (Thermo Fisher Scientific). Gene expression profiling was performed using NanoString® technology. In brief, 50 ng of Total RNA was hybridized at 96° C. overnight with the nCounter® (Mouse immune ‘v1’ Expression Panel NanoString® Technologies). This panel profiles 561 immunology-related mouse gene as well as two types of built-in controls: positive controls (spiked RNA at various concentrations to evaluate the overall assay performance) and 15 negative controls (to normalize for differences in total RNA input). Hybridized samples were then digitally analyzed for frequency of each RNA species using the nCounter SPRINT™ profiler. Raw mRNA abundance frequencies were analyzed using the nSolver™ analysis software 2.5 pack. In this process, normalization factors derived from the geometric mean of housekeeping genes, mean of negative controls and geometric mean of positive controls were used.

TABLE 21
IT-EP of pOMI-mIL12P2A caused an increase in intratumoral levels
of lymphocyte and monocyte cell surface markers in both primary
and distant tumors. Fold change of treated vs. untreated mice values
are shown for measurements taken 7 days after treatment.

| Immune | IT-pOMI-mIL12P2A-EP | | IT-pUMVC3-EP | | Untreated | |
|---|---|---|---|---|---|---|
| Checkpoint | Mean +/− SEM n = 5 | | Mean +/− SEM n = 4 | | Mean +/− SEM n = 3 | |
| Protein RNA | Primary | Distant | Primary | Distant | Primary | Distant |
| CD45 | 11.54 +/− | 3.55 +/− | 1.70 +/− | 1.26 +/− | 1.00 +/− | 1.00 +/− |
| | 1.65 | 0.40 | 0.72 | 0.51 | 0.38 | 0.50 |
| CD3 | 13.16 +/− | 5.30 +/− | 1.26 +/− | 1.09 +/− | 1.00 +/− | 1.00 +/− |
| | 2.95 | 0.72 | 0.38 | 0.32 | 0.22 | 0.40 |
| CD4 | 2.35 +/− | 2.74 +/− | 0.73 +/− | 1.00 +/− | 1.00 +/− | 1.00 +/− |
| | 0.39 | 0.44 | 0.18 | 0.22 | 0.20 | 0.09 |
| CD8 | 16.28 +/− | 4.60 +/− | 1.23 +/− | 1.00 +/− | 1.00 +/− | 1.00 +/− |
| | 3.10 | 0.50 | 0.32 | 0.15 | 0.14 | 0.45 |
| KLRC1 | 14.03 +/− | 5.62 +/− | 1.16 +/− | 1.28 +/− | 1.00 +/− | 1.00 +/− |
| | 2.73 | 0.23 | 0.45 | 0.44 | 0.07 | 0.43 |
| KLRD1 | 4.64 +/− | 4.17 +/− | 1.05 +/− | 1.65 +/− | 1.00 +/− | 1.00 +/− |
| | 1.00 | 0.33 | 0.27 | 0.45 | 0.20 | 0.30 |
| CD11b | 11.13 +/− | 4.17 +/− | 1.55 +/− | 1.11 +/− | 1.00 +/− | 1.00 +/− |
| | 2.39 | 0.48 | 0.52 | 0.40 | 0.42 | 0.34 |

TABLE 22
ΓΓ-EP of pOMI-mIL12P2A caused an increase in intratumoral
levels of INF-γ regulated genes in both primary and distant
tumors. Fold change of treated vs. untreated mice values are shown.

| | IT-pOMI-mIL12P2A-EP | | IT-pUMVC3-EP | | Untreated | |
|---|---|---|---|---|---|---|
| IFN-γ | Mean +/− SEM n = 5 | | Mean +/− SEM n = 4 | | Mean +/− SEM n = 3 | |
| related RNA | Primary | Distant | Primary | Distant | Primary | Distant |
| IFN-γ | 8.63 +/− | 1.80 +/− | 0.76 +/− | 0.98 +/− | 1.00 +/− | 1.00 +/− |
| | 1.38 | 0.44 | 0.22 | 0.43 | 0.15 | 0.29 |
| CD274 | 12.47 +/− | 7.03 +/− | 1.00 +/− | 1.18 +/− | 1.00 +/− | 1.00 +/− |
| (PD-L1) | 2.24 | 2.30 | 0.30 | 0.83 | 0.48 | 0.84 |
| CXCL10 | 3.18 +/− | 2.26 +/− | 0.99 +/− | 1.44 +/− | 1.00 +/− | 1.00 +/− |
| | 0.58 | 0.42 | 0.30 | 0.85 | 0.43 | 0.73 |
| CXCL11 | 5.02 +/− | 3.14 +/− | 0.74 +/− | 1.38 +/− | 1.00 +/− | 1.00 +/− |
| | 0.74 | 0.41 | 0.10 | 0.82 | 0.16 | 0.55 |
| CXCL9 | 5.92 +/− | 3.75 +/− | 1.03 +/− | 1.67 +/− | 1.00 +/− | 1.00 +/− |
| | 0.60 | 0.57 | 0.31 | 1.37 | 0.50 | 0.85 |
| H2A-a | 9.21 +/− | 6.63 +/− | 1.26 +/− | 1.52 +/− | 1.00 +/− | 1.00 +/− |
| | 1.86 | 2.21 | 0.36 | 0.99 | 0.61 | 1.28 |
| H2k-1 | 4.23 +/− | 3.71 +/− | 1.06 +/− | 1.42 +/− | 1.00 +/− | 1.00 +/− |
| | 1.02 | 0.68 | 0.19 | 0.52 | 0.54 | 0.87 |
| IRF 1 | 4.18 +/− | 2.72 +/− | 1.09 +/− | 1.28 +/− | 1.00 +/− | 1.00 +/− |
| | 0.28 | 0.46 | 0.28 | 0.93 | 0.45 | 0.78 |
| PDCD1 | 3.80 +/− | 2.78 +/− | 1.13 +/− | 1.18 +/− | 1.00 +/− | 1.00 +/− |
| (PD-1) | 0.48 | 0.84 | 0.25 | 0.37 | 0.28 | 0.56 |
| Stat 1 | 3.51 +/− | 3.47 +/− | 1.04 +/− | 1.36 +/− | 1.00 +/− | 1.00 +/− |
| | 0.28 | 0.68 | 0.26 | 0.79 | 0.48 | 0.79 |
| TAP 1 | 3.80 +/− | 2.84 +/− | 1.17 +/− | 1.36 +/− | 1.00 +/− | 1.00 +/− |
| | 0.48 | 0.37 | 0.27 | 0.85 | 0.50 | 0.97 |
| CCL5 | 24.47 +/− | 14.59 +/− | 2.21 +/− | 1.48 +/− | 1.00 +/− | 1.00 +/− |
| | 7.81 | 2.97 | 0.72 | 0.40 | 0.29 | 0.40 |
| CCR5 | 11.29 +/− | 3.70 +/− | 1.31 +/− | 1.21 +/− | 1.00 +/− | 1.00 +/− |
| | 2.72 | 0.70 | 0.42 | 0.42 | 0.27 | 0.40 |
| GZMA | 11.08 +/− | 4.60 +/− | 1.43 +/− | 2.05 +/− | 1.00 +/− | 1.00 +/− |
| | 1.18 | 0.96 | 0.53 | 0.91 | 0.23 | 0.22 |
| GZMB | 3.11 +/− | 2.11 +/− | 0.68 +/− | 1.47 +/− | 1.00 +/− | 1.00 +/− |
| | 0.83 | 0.10 | 0.22 | 0.67 | 0.33 | 0.47 |
| PRF1 | 8.21 +/− | 2.06 +/− | 1.0 +/− | 1.13 +/− | 1.00 +/− | 1.00 +/− |
| | 2.27 | 0.26 | 0.32 | 0.45 | 0.23 | 0.39 |

Additional NanoString® gene expression analysis of extracts from primary, treated and distant, untreated tumors in the 4T1 and MC-38 tumor models after pOM1-mIL12P2A electroporation revealed similar upregulation of lymphocyte and monocyte cell surface markers as well as INF-γ-regulated genes, indicating that these effects of IL-12 on the tumor microenvironment are generalizable to multiple mouse tumor models.

Gene expression analysis of tissue from primary, treated and distant, untreated tumors corroborate flow cytometric analysis showing a robust increase in tumor TIL with IT-EP of pOM1P2A-mIL12. In addition, an increase in interferon gamma-regulated genes suggest induction of an immunostimulatory environment within the tumors. A significant increase in expression of checkpoint proteins indicate that IT-pOMI-mIL12P2A-EP could increase the substrate for the action of checkpoint inhibitors used in combination.

Seven days after Intratumoral electroporation of B16-F10 tumors with pOMI-PIIM using 400 V/cm, and 8 10-ms pulses, tumors were surgically removed and RNA extracted for the analysis of gene expression changes mediated by the combination of IL-12 and FLT3L-NYESO1 intratumoral expression.

TABLE 23
IT-EP of pOMI-PIIM caused an increase in intratumoral levels
of lymphocyte and monocyte cell surface markers, INF-γ
regulated genes, and antigen presentation machinery in primary
(treated) tumors. Fold change of treated vs. untreated mice
values are shown for measurements taken 7 days after treatment.

| TIL, INF-γ, or APM | EP/pOMI-PIIM | EP/pUMVC3 |
|---|---|---|
| related RNA | Mean n = 4 | Mean n = 3 |
| CD3e | 12.44 | 1.91 |
| CD4 | 5.90 | 2.63 |
| CD8 | 10.02 | 2.12 |
| KLRC1 | 17.43 | 2.00 |
| KLRD1 | 5.94 | 2.33 |
| CD274 | 42.56 | 2.52 |
| IFN-γ | 10.81 | 0.55 |
| CCL9 | 48.53 | 8.59 |
| CCL10 | 9.53 | 2.37 |
| CCL11 | 9.26 | 3.05 |
| IRF1 | 17.24 | 4.35 |
| PCDC1 | 5.25 | 1.24 |
| STAT1 | 13.26 | 2.40 |
| CCL5 | 72.09 | 5.18 |
| CCR5 | 25.61 | 3.34 |
| PRF1 | 16.27 | 2.36 |
| CIITA | 68.90 | 8.03 |
| H2-0b | 36.14 | 2.19 |
| H2-Aa | 53.34 | 6.96 |
| H2-k1 | 8.53 | 2.27 |
| H2-Ab1 | 88.17 | 8.93 |
| H2-eb1 | 49.30 | 7.55 |
| TAP1 | 12.58 | 2.81 |
| TAP1bp | 10.27 | 2.95 |
| CD74 | 54.65 | 7.60 |
| CD11b | 24.15 | 3.15 |

Intratumoral expression of IL-12 protein after electroporation of a plasmid for expression of multiple genes still induced significant changes in gene expression associated with a robust adaptive immune response. The addition of intratumoral expression of the FLT3L-NYESO1 fusion protein induced a measurable increase in expression of gene associated with antigen presentation in the treated tumors.

TABLE 24
IT-EP of pOMI-PIIM caused an increase in intratumoral levels
of lymphocyte and monocyte cell surface markers and INF-
γ regulated genes in distant (untreated) tumors. Fold
change of treated vs. untreated mice values are shown.

| TIL and IFN-γ | pOMI-PIIM | IT-pUMVC3-EP |
|---|---|---|
| related RNA | Mean n = 4 | Mean n = 3 |
| CD45 | 8.75 | 3.00 |
| CD8 | 4.79 | 2.12 |
| KLRC1 | 6.54 | 1.88 |
| CD11b | 8.28 | 2.64 |
| CD274 (PD-L1) | 13.97 | 2.36 |
| CXCL9 | 20.00 | 5.05 |
| CXCL10 | 4.33 | 1.78 |
| H2a-a | 19.28 | 3.61 |
| H2k-1 | 4.34 | 1.76 |
| IRF1 | 7.01 | 1.53 |
| STAT1 | 7.18 | 1.86 |
| TAP1 | 5.96 | 1.90 |
| CCL5 | 23.40 | 3.83 |
| CCR5 | 6.89 | 2.55 |

Intratumoral electroporation of a plasmid encoding both mIL-12 and FLT3L-NYESO1 demonstrated significant changes in intratumoral gene expression consistent with increasing both local and systemic anti-tumoral immunity and corroborate the strong effect of this therapy on controlling growth of both primary, treated and distant, untreated tumors in this mouse model (Table 17 and FIG. 3).

Intratumoral electroporation of an OMI plasmid encoding human FLT3L-NYESO1 fusion protein alone also had effects on tumor regression and changes to the immune phenotype of tumor TIL.

TABLE 25
IT-EP of pOMI-FLT3L-NYESO1 plasmid reduced tumor growth.
Subcutaneous B16-F10 tumors were electroporated once at 400 V/cm,
8 10 ms pulses with acupuncture needles after plasmid injection.
Tumor measurements on Day 6 after treatment are shown.

| | Tumor volume (mm³) |
|---|---|
| Treatment | Mean +/− SEM n = 5 |
| Untreated | 273.8 +/− 35.7 |
| EP/pUMVC3 (empty vector) | 380.4 +/− 84.7 |
| EP/pOMI-FLT3L-NYESO1 | 127.1 +/− 13.2 |
| EP/pOMIP2A-IL12 | 69.4 +/− 16.4 |

TABLE 26
Changes INF-γ related gene expression in treated
tumors after IT-EP of pOMI-FLT3L-NYESO1 as measured
by NanoString ® in tumor extracts. Fold
change of treated vs. untreated mice values are shown.

| IFN-γ | IT-EP pUMVC3 | IT-EP pOMI-FLT3L-NYESO1 |
|---|---|---|
| related RNA | Mean +/− SEM n = 3 | Mean +/− SEM n = 5 |
| CXCL9 | 1.00 +/− 0.07 | 3.68 +/− 0.42 |
| CXCL10 | 1.00 +/− 0.02 | 1.80 +/− 0.17 |
| CXCL11 | 1.00 +/− 0.35 | 2.29 +/− 0.41 |
| CD274 | 1.00 +/− 0.28 | 3.31 +/− 0.55 |
| IRF1 | 1.00 +/− 0.07 | 2.31 +/− 0.16 |
| STAT1 | 1.00 +/− 0.13 | 2.46 +/− 0.25 |

TABLE 27
Changes in antigen presentation machinery (APM) gene expression
was detected in treated tumors after IT-EP of pOMI-FLT3L-
NYESO1 as measured by NanoString ® in tumor extracts.
Fold change of treated vs. untreated mice values are shown.

| | IT-EP pUMVC3 | IT-EP pOMI-FLT3L-NYESO1 |
|---|---|---|
| APM RNA | Mean +/− SEM n = 3 | Mean +/− SEM n = 5 |
| H2-Ob | 1.00 +/− 0.24 | 2.09 +/− 0.48 |
| H2-Aa | 1.00 +/− 0.29 | 4.41 +/− 0.78 |
| H2-K1 | 1.00 +/− 0.21 | 2.20 +/− 0.16 |
| H2-Ab1 | 1.00 +/− 0.22 | 4.78 +/− 0.82 |
| H2-Eb1 | 1.00 +/− 0.22 | 3.74 +/− 0.50 |
| TAP1 | 1.00 +/− 0.08 | 2.63 +/− 0.25 |
| TAPbp | 1.00 +/− 0.11 | 2.61 +/− 0.23 |
| CD74 | 1.00 +/− 0.22 | 4.71 +/− 0.81 |
| CCR7 | 1.00 +/− 0.09 | 2.08 +/− 0.33 |
| CD11b | 1.00 +/− 0.18 | 2.22 +/− 0.27 |

TABLE 28
Changes in co-stimulatory gene expression in treated
tumors after IT-EP of pOMI-FLT3L-NYESO1 as measured
by NanoString in tumor extracts. Fold change of
treated vs. untreated mice values are shown.

| | | IT-EP pOMI-FLT3L- |
|---|---|---|
| Co-stimulatory | IT-EP pUMVC3 | NYESO1 |
| RNA | Mean +/− SEM n = 3 | Mean +/− SEM n = 5 |
| CD80 | 1.00 +/− 0.12 | 2.01 +/− 0.35 |
| CD40 | 1.00 +/− 0.18 | 3.15 +/− 0.52 |
| CTLA4 | 1.00 +/− 0.06 | 3.11 +/− 0.47 |
| CD274 | 1.00 +/− 0.28 | 3.31 +/− 0.55 |
| ICAM1 | 1.00 +/− 0.33 | 2.67 +/− 0.55 |

TABLE 29
Changes in T cell and Natural Killer (NK) cell-related gene
expression in treated tumors after IT-EP of pOMI-FLT3L-NYESO1
as measured by NanoString ® in tumor extracts.
Fold change of treated vs. untreated mice values are shown.

| | | IT-EP pOMI-FLT3L- |
|---|---|---|
| T and NK | IT-EP pUMVC3 | NYESO1 |
| cell RNA | Mean +/− SEM n = 3 | Mean +/− SEM n = 5 |
| KLRC1 | 1.00 +/− 0.37 | 2.84 +/− 0.40 |
| KLRD1 | 1.00 +/− 0.11 | 3.91 +/− 0.74 |
| CD3e | 1.00 +/− 0.38 | 3.57 +/− 0.70 |
| CD8a | 1.00 +/− 0.36 | 2.03 +/− 0.38 |
| CD4 | 1.00 +/− 0.10 | 2.08 +/− 0.36 |

Intratumoral electroporation of a plasmid for expression Flt3L-NYESO1 fusion protein demonstrated measurable effects on immune cell and APM related gene expression in the absence of IL-12 co-expression indicating that Flt3L-NYESO1 has independent effects on intratumoral immune modulation when introduced by IT-EP (Tables 26,27,28,29).

### XIV. Detection of Host Response to Tracking Antigen by Flow Cytometry

In order to test for host response to electroporation of plasmids encoding a tracking antigen fused to Flt3L, B16-F10 tumors were electroporated with pOMI-mIL12P2A-FLT3L-OVA and the host response to the OVA antigen was measured. Mice were injected with 1 million B16-F10 cells on the right flank. Seven days later, tumors were electroporated with pOMI-mIL12P2A-FLT3L-OVA, empty vector, or left untreated. Electroporation was done using a generator with Electrochemical Impedence Sensing (EIS), see, e.g., WO2016161201, 400 V/cm, 8 10-ms pulses. As with pOMI-PIIM containing mouse IL-12 (Table 17), tumor regression was observed with pOMI-mIL12P2A-FLT3L-OVA in this experiment.

Detection of tracking antigen-specific CD8+ T cells in mouse was tested in inguinal lymph nodes 7 days after IT-EP of a plasmid encoding mIL12 and FLT3L-OVA fusion proteins into tumors.

Mice were sacrificed; inguinal lymph nodes were excised, mashed in PBS+2% FBS+1 mM EDTA (PFB) and then strained through a 70 micro filter. Cells were pelleted in a centrifuge at 300×g at 4° C. and washed in PFB, and counted on a Cellometer (Nexcelom).

Lymph node cell pellets were gently re-suspended in PFB with Fc block (BD Biosciences 553142). Cells were then mixed with a solution of SIINFEKL tetramer (MBL), according to the manufacturers instruction and incubated for 10 minutes at room temperature. Antibody staining cocktails containing the following: Live/Dead Aqua (Thermo Fisher L34966), Anti-CD3 (Biolegend 100228), anti-CD19 (Biolegend 115555), anti-CD127 (Biolegend), anti-CD8a (MBL D271-4), anti-CD44 (Biolegend 103028), anti-PD-1 (Biolegend 109110), anti-CD4 (Biolegend 100547), anti-KLRG1 (138419), anti-CD62L (Biolegend 104448) were added and incubated at 4° for 30 minutes. Cells were washed with PFB. Cells were fixed in PFB with 1% paraformaldehyde for 1 minute on ice. Cells were washed 3 times with PFB, and analyzed by flow cytometry (LSR Fortessa X-20).

TABLE 30
Detection of host T cells reactive to ovalbumin tracking
antigen after IT-EP of pOMI-mIL12P2A-FLT3L-OVA as compared
to pUMVC3 empty vector into B16-F10 subcutaneous tumors.

| | Frequency of | Frequency of |
|---|---|---|
| Plasmid introduced | CD44⁺SIINFEKL | SIINFEKL |
| by IT-EP | tetramer⁺CD8⁺ T cells | tetramer⁺CD8⁺ T cells |
| Untreated n = 3 | 0.0003 +/− 0.0003 | 0.0067 +/− 0.0018 |
| pUMVC3 n = 4 | 0.0026 +/− 0.0003 | 0.0100 +/− 0.0027 |
| pOMI-mIL12-hFLT3L- | 0.4050 +/− 0.2457 | 0.2958 +/− 0.0582 |
| OVA n = 6 | | |

Using OVA as a surrogate tracking antigen in mice, we demonstrate that we can readily detect circulating T cells directed against the tracking antigen, which was electroporated into tumor as a FLT3L-fusion protein (Table 30).

XV. Introduction of Plasmids by Hydrodynamic Injection into Mouse Tail Vein

The in vivo activity of FLT3L fusion proteins expressed from OMI plasmids was tested by hydrodynamic injection of 5 μg of plasmids into the tail vein of C57Bl/6J mice. Seven days later, mice were sacrificed; the spleens were excised, weighed, and dissociated for analysis of changes in cell composition by flow cytometry.

Splenocytes were isolated as described above, washed with PFB and re-suspended in PFB with Fc block (BD Biosciences 553142) and incubated for 10 minutes at room temp. Antibody cocktails containing the following were added: Anti NK1.1 (Biolegend108731), Live/Dead Aqua (Thermo Fisher L34966), anti-CD4 (Biolegend 100547), anti-F4/80 (Biolegend 123149), anti-CD19 (Biolegend 115555), Anti-I-A/I-E (Biolegend 107645), Anti-CD8 (MBL International D271-4), anti-CD80 (Biolegend 104722), anti-CD3 (Biolegend 117308), anti-CD40 (Biolegend 124630), anti-GR-1 (Biolegend 108424), anti-CD11c (Biolegend 117324), anti-CD86 (Biolegend 105024, anti-CD11b (Biolegend 101212). Incubate at 37° C. Cells were washed 3 times with PFB, and analyzed by flow cytometry (LSR Fortessa X-20).

TABLE 31
Effect of systemic exposure to pOMIP2A-FLT3L and pOMIP2A-
FLT3L-NYESO1 plasmids introduced by tail vein injection.

| | | Absolute | CD11c⁺ frequency |
|---|---|---|---|
| | Spleen weight | CD11c⁺ cell | of parent CD3 |
| | (grams) | number; Mean × | CD3⁻CD19⁻NK1.1; |
| Injected | Mean +/− | 10⁶ +/− SEM, | Mean percent +/− |
| Plasmid | SEM, n = 6 | n = 6 | SEM, n = 6 |
| None | 0.085 +/− 0.005 | 1.82 | 7.68 +/− 0.66 |
| pUMVC3 | 0.090 +/− 0.006 | 2.75 | 12.11 +/− 0.08 |
| empty | | | |
| vector | | | |
| OMIP2A- | 0.123 +/− 0.009 | 5.26 | 31.75 +/− 2.88 |
| FLT3L- | | | |
| NYESO1 | | | |
| OMIP2A- | 0.141 +/− 0.011 | 5.42 | 37.60 +/− 3.22 |
| FLT3L | | | |

Introduction of plasmids encoding human FLT3L or human FLT3L fused to a portion of the NY-ESO-1 proteins (80-180 aa) lead to an increase in CD11c⁺ dendritic cells (DC) in the spleen (Table 31). Moreover, the majority of these DC demonstrated high levels of MI-IC Class II indicating that they are mature DCs. In addition, a portion of these DCs demonstrated higher levels of cell surface CD86 expression, indicating they were activated.

These data are consistent with exposure to active FLT3 ligand being expressed from these plasmids and leading to DC maturation and activation in vivo (Maraskovsky et al., 2000. Blood 96:878)

XVI. Maturation of Human Dendritic Cells In Vitro with Flt3L-Fusion Proteins

Human Flt3L-NY-ESO1 fusion proteins expressed from pOMI-PIIM were tested for the ability to mature ex-vivo cultured, immature human DCs. To accomplish this, DCs were cultured using standard protocols (Pollack SM J R et al., (2013) Tetramer Guided Cell Sorter Assisted Production of NY-ESO-1 Specific Cells for the Treatment of Synovial Sarcoma and Myxoid Round Cell Liposarcoma. Connective Tissue Oncology Society Meeting), first isolating monocytes from healthy donor peripheral blood mononuclear cells (PBMCs), and then culturing those monocytes in serum free media with GM-CSF and IL-4 for 5-7 days prior to treatment. These immature DCs were then left untreated, or treated with media conditioned by HEK 293 cells previously transfected with either pOMI-PIIM, an empty negative control vector (EV), or a control vector with a mutated gene for expression of a Flt3L-NYESO1 fusion protein which is unable to bind to Flt3 and therefore should be inactive (Flt3L-NY-ESO-1 (H8R) as well as recombinant purified FLT3L used as a positive control for 48 hours.

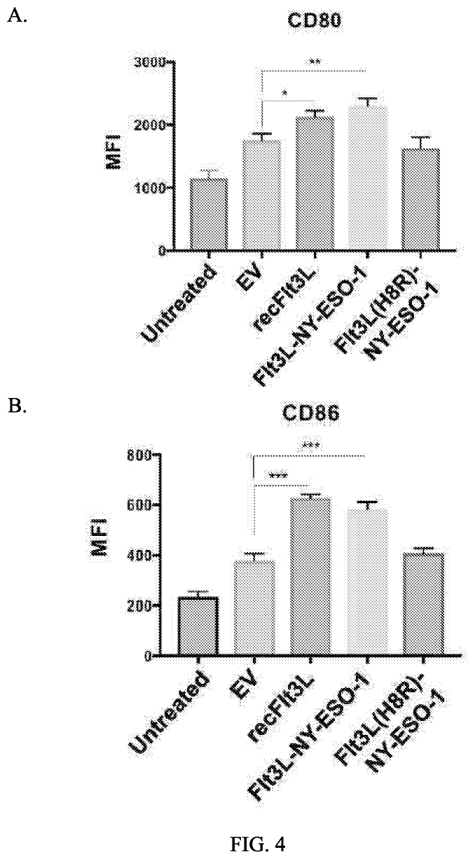

As measured by flow cytometry, CD80 and CD86 cell surface markers were used as the primary metrics for FLT3L-mediated DC activity on all cells that were CD11c⁺DC-SIGN⁺. Conditioned media from cells transfected with pOMI-PIIM had significantly more induction of both CD80 and CD86 compared with either media from cells with the empty vector or the vector encoding the Flt3L(H8R) inactive mutant (FIG. 4). Culture supernatants from cells transfected with pOMI-PIIM plasmid had similar activity in comparison to the recombinant Flt3L protein used as a positive control. These studies were repeated ensuring their reproducibility. Some non-specific induction of CD80/CD86 expression was observed with addition of control supernatants (not containing any plasmid derived proteins) as compared to untreated.

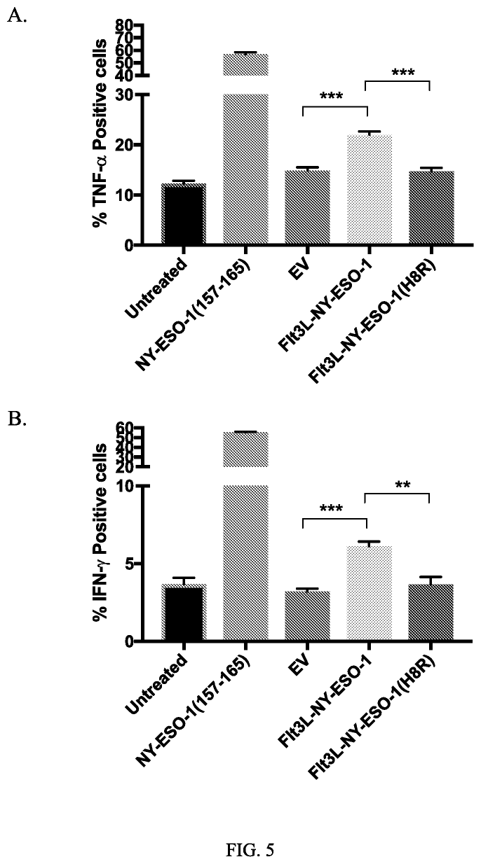

Stimulation of NY-ESO-1 specific T cells by co-culture with Flt3L-NYESO transduced DCs. Pre-established NY-ESO-1 specific CTL lines were stimulated using transduced DCs (described in section XVI) and then analyzed by flow cytometry staining for intracellular cytokines, TNFα and INF-γ. These data show that DCs pulsed with plasmid-derived Flt3L-NY-ESO-1, but not an inactive mutant (Flt3L-NY-ESO-1 (H8R)), are able to activate NY-ESO-1 specific CTL lines (FIG. 5).

These data showed that human Flt3L-NY-ESO1 fusion protein expressed from pOMI-PIIM could induce maturation of primary immature human dendritic cells.

That which is claimed:

1. An expression vector comprising the nucleic acid sequence of SEQ ID NO: 1.

2. An expression vector comprising a nucleic acid encoding a polypeptide comprising an amino acid having at least 70% identity to the amino acid sequence of SEQ ID NO: 9.

3. The expression vector of claim 2, wherein the polypeptide comprises the amino acid sequence of SEQ ID NO: 9.

4. The expression vector of claim 2 or 3, wherein the nucleic acid comprises a nucleotide sequence having at least 70% identity to the nucleotide sequence of SEQ ID NO: 8.

5. The expression vector of claim 4, wherein the nucleic acid comprises the nucleotide sequence of SEQ ID NO: 8.

6. The expression vector of claim 4 or 5, wherein the nucleic acid is operably linked to a nucleic acid encoding a P2A translation modification element and a nucleic acid encoding a FLT-3L peptide fused to at least one antigen.

7. The expression vector of claim 6, wherein the antigen is selected from the group consisting of: NYESO-1, OVA, RNEU, MAGE-A1, MAGE-A2, Mage-A10, SSX-2, Melan-A, MART-1, Tyr, Gp100, LAGE-1, Survivin, PRS pan-DR, CEA peptide CAP-1, OVA, HCV-NS3, TERT, WT1, PSMA, and an HPV vaccine peptide.

8. The expression vector of claim 7, wherein the antigen is NYESO-1.

9. The expression vector of any one of claims 2-8, wherein the nucleic acid is operably linked to a CMV promoter.

10. The expression vector of any one of claims 2-9, wherein the polypeptide comprises an amino acid sequence having at least 70% identity to the amino acid sequence of SEQ ID NO: 11.

11. The expression vector of claim 10, wherein the polypeptide comprises the amino acid sequence of SEQ ID NO: 11.

12. The expression vector of claim 10 or 11, wherein the nucleic acid comprises a nucleotide sequence having at least 70% identity to the nucleotide sequence of SEQ ID NO: 10.

13. The expression vector of claim 12, wherein the nucleic acid comprises the nucleotide sequence of SEQ ID NO: 10.

14. The expression vector of claim 12 or 13, wherein the nucleic acid is operably linked to a CMV promoter.

15. The expression vector of claim 14, wherein the expression vector comprises a nucleotide sequence having at least 70% identity to the nucleotide sequence of SEQ ID NO: 12.

16. The expression vector of claim 15, wherein the expression vector comprises the nucleotide sequence of SEQ ID NO: 12.

17. A method of treating a tumor in a subject, comprising delivering the expression vector any one of claims 1-16 into the tumor using at least one intratumoral electroporation pulse.

18. The method of claim 17, wherein the intratumoral electroporation pulse has a field strength of about 200 V/cm to about 1500 V/cm.

19. The method of claim 17 or 18, wherein the subject is a human.

20. The method of any one of claims 17-19, wherein the tumor is selected from the group of melanoma, triple negative breast cancer, Merkel Cell Carcinoma, CTCL, and head and neck squamous cell carcinoma.

21. The method of any one of claims 17-20, wherein the electroporation pulse is delivered by a generator capable of electrochemical impedance spectroscopy.

22. A method of treating a tumor in a subject, comprising administering at least one low voltage intratumoral electroporation (IT-EP) treatment that delivers an expression vector comprising:
a. the nucleotide sequence of SEQ ID NO: 1, SEQ ID NO: 8, SEQ ID NO: 10, or SEQ ID NO: 12;
b. a nucleotide sequence having at least 70% identity to the nucleotide sequence of SEQ ID NO: 1, SEQ ID NO: 8, SEQ ID NO: 10, or SEQ ID NO: 12;
c. a nucleotide sequence encoding a polypeptide comprising the amino acid sequence of SEQ ID NO: 9 or SEQ ID NO: 11; or
d. a nucleotide sequence encoding a polypeptide having at least 70% identity to the amino acid sequence of SEQ ID NO: 9 or SEQ ID NO: 11.

23. The method of claim 22, wherein the IT-EP treatment comprises a field strength from about 200 V/cm to about 500 V/cm and a pulse length of about 100 μs to about 50 ms.

24. The method of claim 23, wherein the treatment is one IT-EP treatment and comprises a field strength of about 350-450 V/cm and a pulse length of about 10 ms.

25. The method of claim 24, wherein the treatment is one IT-EP treatment and comprises a field strength of about 400 V/cm and a pulse length of about 10 ms.

26. The method of any one of claims 17-25, wherein the treatment comprises 1-10 10 ms electroporation pulses.

27. The method of claim 26, wherein the treatment comprises 5-10 10 ms electroporation pulses.

28. The method of claim 27, wherein the treatment comprises 8 10 ms electroporation pulses.

29. The method of any one of claims 22-28, wherein the treatment results in one or more or all of the following when compared to low voltage IT-EP treatment with an IL-12 encoding plasmid containing an IRES motif:
a. at least 3.6 times higher intratumoral expression of IL-12;
b. a lower mean tumor volume in a treated tumor lesion;
c. a lower mean tumor volume in an untreated contralateral tumor lesion;
d. a higher influx of lymphocytes into the tumor;
e. an increase of circulating tumor-specific CD8+ T cells;
f. an increase of lymphocyte and monocyte cell surface marker expression in the tumor; and
g. an increase in mRNA levels of INF-γ related genes of Tables 23 and 24.

30. The expression vector of any of claims 1-16 for use in treating a tumor in a subject wherein treating comprises delivering the expression vector into the tumor using at least one intratumoral electroporation pulse.

31. The expression vector of claim 30 wherein the intratumoral electroporation pulse comprises at least one low voltage intratumoral electroporation (IT-EP) treatment.

32. The expression vector of claim 31, wherein the IT-EP treatment comprises at a field strength from 200 V/cm to 500 V/cm and a pulse length of about 100 μs to about 50 ms.

33. The expression vector of claim 32 wherein the treatment is one IT-EP treatment and comprises a field strength of at 350-450 V/cm and a pulse length of about 10 ms.

34. The expression vector of claim 33 wherein the treatment is one IT-EP treatment and comprises a field strength of about 400 V/cm and a pulse length of about 10 ms.

35. The expression vector of any of claims 30-34 wherein the treatment comprises 1-10 10 ms electroporation pulses.

36. The expression vector of claim 35 wherein the treatment comprises 5-10 10 ms electroporation pulses.

37. The expression vector of claim 36 wherein the treatment comprises 8 10 ms electroporation pulses.
